(19) United States
(12) Patent Application Publication
(10) Pub. No.: US 2008/0318255 A1
(43) Pub. Date: Dec. 25, 2008

(54) Fluorescent Labeled Sphingosines

(76) Inventors: Andreas Billich, Modling (AT); Peter Ettmayer, Wien (AT); Diana Mechtcheriakova, Wien (AT); Peter Nussbaumer, Maria Enzersdorf (AT); Alexander Wlachos, Wien (AT)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 10/573,602

(22) PCT Filed: Sep. 28, 2004
(86) PCT No.: PCT/EP2004/010862

§ 371 Date: May 4, 2006

Related U.S. Application Data
(60) Provisional application No. 60/507,065, filed on Sep. 29, 2003.
(60) Provisional application No. 60/507,064, filed on Sep. 29, 2003.

(51) Int. Cl. G01N 33/573; C07C 215/10; C07F 9/32; C07D 311/80

(52) U.S. Cl. 435/7.72; 564/506; 564/15; 548/113; 549/391

(57) ABSTRACT

The present invention relates to a method (assay) for determining the activity of an enzyme selected from the group consisting of a sphingosine kinase and a phosphatase involved in the sphingolipid pathway by use of a labeled sphingosine.

The present invention relates to labeled sphingosines, e.g. fluorescent labeled sphingosines, e.g. for use in a method (assay) for determining the activity of an enzyme such as a sphingosine kinase and a phosphatase involved in the sphingolipid pathway.

Sphingosine-1-phosphate (SPP) is an important signaling molecule with both intra- and extracellular actions (see e.g. Baumruker T. et al., Semin. Immunol. 2002, 14, 57-63). SPP is formed from sphingosine (SP) by sphingosine kinases (SPHKs) in the presence of a phosphate source, e.g. adenosine triphosphate (ATP), guanosine triphosphate (GTP). For studies on the function and subcellular localization of both SP and SPP, determination of the bioactivity of SPHKs, e.g. by determination of an SP/SPP ratio may be essential. Sphingosines which are acylated at the amino group in position 2 are recognized by ceramide kinases and in the presence of a phosphate source, e.g. adenosine triphosphate (ATP) or guanosine triphosphate (GTP), these sphingosines are phosphorylated.

We now have surprisingly found a method for determining the activity of an enzyme selected from the group consisting of a sphingosine kinase and a phosphatase involved in the sphingolipid pathway, such as e.g. a sphingosine phosphate phosphatase or a lipid phosphate phosphatase, in a sample, which may be carried out easily and effective.

In one aspect the present invention provides the use of a labeled sphingosine or a labeled sphingosine phosphate for determining whether an activity of an enzyme selected from the group consisting of a sphingosine kinase and a phosphatase involved in the sphingolipid pathway, such as e.g. a sphingosine phosphate phosphatase and a lipid phosphate phosphatase, is present in a sample or not, or determining the extent of said activity.

In another aspect the present invention provides a method for determining whether an activity of an enzyme selected from the group consisting of a sphingosine kinase and a phosphatase involved in the sphingolipid pathway, such as e.g. a sphingosine phosphate phosphatase and a lipid phosphate phosphatase, is present in a sample or not, or determining the extent of said activity comprising the steps of
a. contacting living cells comprised in an appropriate culture medium with a labeled sphingosine for a predetermined period of time so that an enzymatic product can be formed,
b. separating the enzymatic product formed in step a., and
c. determining the amount of enzymatic product formed.

Separation of the enzymatic product formed in step b. may be carried out as appropriate, e.g. by adding to the mixture obtained in step a. an aqueous buffer solution and organic solvent which is able to form two phases in combination with water and separating the phases obtained, e.g. after extraction.

Any cell type which has the ability to uptake enzymes from the group consisting of a sphingosine kinase, a sphingosine phosphate phosphatase and a lipid phosphate phosphatase can be used in a method of the present invention. Preferably human cells are used, e.g. human endothelial cells, such as e.g. human umbilical vein endothelial cells (HUVEC).

Cells are used as living cells in an appropriate medium, e.g. a medium suitable for culturing cells, such as human cells, e.g. human endothelial cells, including HUVEC.

A labeled sphingosine used in the described cell based assay is a compound of formula I, as described herein.

For the determination of sphingosine kinase activity the labeled sphingosine used is an SP, namely an unphosphorylated sphingosine, whereas for the determination of sphingosine phosphate phosphatase and/or lipid phosphate phosphatase activity the labeled sphingosine used is n SPP, namely an 1-phosphate-sphingosine.

In a further aspect the present invention provides a method for determining whether a sphingosine kinase activity is present or not, or determining the extent of a sphingosine kinase activity in a sample, comprising the steps of
A. contacting a labeled unphosphorylated sphingosine with
   a phosphate source, and
   a sample which sample optionally comprises a sphingosine kinase for a predetermined period of time so that an enzymatic product can be formed,
B. adding to the mixture of step A. an aqueous buffer solution and organic solvent which is able to form two phases in combination with water,
C. separating the phases obtained in step b., e.g. after extraction,
D. determining the amount of enzymatic product in the aqueous phase obtained in step c.

A sphingosine as used herein includes naturally occurring sphingosines and sphingosine phosphates, and sphingosines and sphingosine phosphates which are chemically modified and chemically different from natural sphingosines or sphingosine phosphates, but which are still recognised as a substrate by an enzyme selected from the group consisting of a sphingosine kinase, or, respectively, a sphingosine phosphate phosphatase or a lipid phosphate phosphatase.

Chemically modified sphingosines and sphingosine phosphates may be optimized as regards to various parameters, such as e.g. enzyme reaction rates or solubility, and may be valuable tools in investigations on SP, SPP and SPHKs and may be produced according, e.g. analogously, to a method as conventional or as described herein.

A labeled sphingosine useful in a method according to the present invention includes e.g. a compound of formula

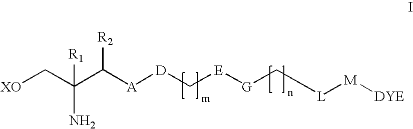

wherein
R₁ is H or (C₁₋₄)alkyl,
R₂ is H, OH or oxo, e.g. H or OH,

### X is H or (HO)₂PO,

A-D, E-G and L-M independently of each other is a group

### CH₂—CH₂, CH═CH, C≡C,

CH₂-phenyl, phenyl-CH₂, CH₂—CH₂-phenyl,
e.g. including a group of formula

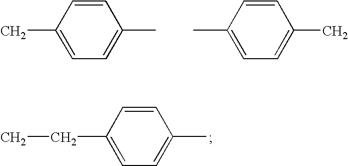

CH₂—NH, CH₂—N((C₁₋₄)alkyl), NH—CH₂, N((C₁₋₄)alkyl)-CH₂, O—CH₂, CH₂—O, phenyl-O, O-phenyl, CH₂-phenyl-O, e.g. including a group of formula

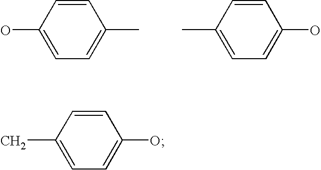

O—CO, CO—O, CO—NH, NH—CO, CO—N((C₁₋₄)alkyl), N(C₁₋₄)alkyl)-CO, NH—SO₂, SO₂—NH, N((C₁₋₄)alkyl)-SO₂, or
one group out of A-D, E-G and L-M is absent,
m is a number selected from 0 to 12,
n is a number selected from 0 to 12,
m plus n is a number selected from 0 to 14,
the group DYE is a group selectively detectable in a compound of formula I by physical
means,
with the proviso that
at least one of E-G and L-M is selected from the group consisting of CH₂—NH, CH₂—N((C₁₋₄)alkyl), CH₂—O, phenyl-O, O—CO, CO—O, CO—NH, NH—CO, CO—N((C₁₋₄)alkyl), N(C₁₋₄)alkyl)-CO, NH—SO₂, N((C₁₋₄)alkyl)-SO₂.

In another aspect the present invention provides a compound of formula

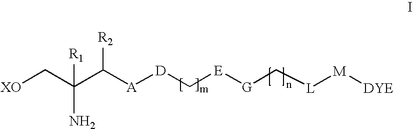

wherein
R₁ is H or (C₁₋₄)alkyl,
R₂ is H, OH or oxo, e.g. H or OH,

### X is H or (HO)₂PO,

A-D, E-G and L-M independently of each other is a group

### CH₂—CH₂, CH═CH, C≡C,

CH₂-phenyl, phenyl-CH₂, CH₂—CH₂-phenyl,
e.g. including a group of formula

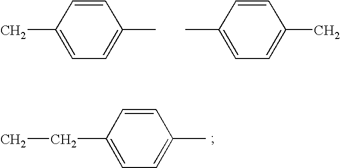

CH₂—NH, CH₂—N((C₁₋₄)alkyl), NH—CH₂, N((C₁₋₄)alkyl)-CH₂, O—CH₂, CH₂—O, phenyl-O, O-phenyl, CH₂-phenyl-O, e.g. including a group of formula

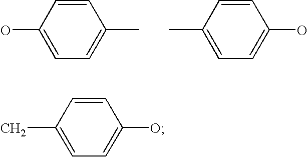

O—CO, CO—O, CO—NH, NH—CO, CO—N((C₁₋₄)alkyl), N(C₁₋₄)alkyl)-CO, NH—SO₂, SO₂—NH, N((C₁₋₄)alkyl)-SO₂,
or one group out of A-D, E-G and L-M is absent,
m is a number selected from 0 to 12,
n is a number selected from 0 to 12,
and m plus n is a number selected from 0 to 14,
the group DYE is a group selectively detectable in a compound of formula I by physical
means,
with the proviso that
at least one of E-G and L-M is selected from the group consisting of CH₂—NH, CH₂—N((C₁₋₄)alkyl), CH₂—O, phenyl-O, O—CO, CO—O, CO—NH, NH—CO, CO—N((C₁₋₄)alkyl), N(C₁₋₄)alkyl)-CO, NH—SO₂, N((C₁₋₄)alkyl)-SO₂, and
a compound of formula

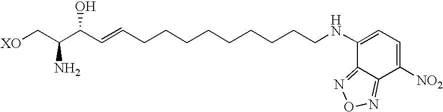

wherein X is as defined above, is excluded.

In a method of the present invention preferably for the determination of
a sphingosine kinase activity, the labeled sphingosine used is a compound of formula I wherein X is H,
a sphingosine phosphate phosphatase and/or a lipid phosphate phosphatase activity the labeled sphingosine used is a compound of formula I, wherein X is (HO)₂PO.

If not otherwise defined herein
acyl includes alkylcarbonyl, cycloalkylcarbonyl, arylcarbonyl, heterocyclylcarbonyl,
alkyl includes (C₁₋₄)alkyl,
cycloalkyl includes (C₃₋₈)cycloalkyl,
aryl includes (C₆₋₁₈)aryl, e.g. phenyl, naphthyl,
heterocyclyl includes a heterocyclic ring (system) having 5 or 6 ring members and 1 to 4 heteroatoms selected from the group consisting of N, S, O.

A labeled sphingosine used in the methods of the present invention comprises a sphingosine labeled with a group selectively detectable by physical means, e.g. a group selectively detectable by fluorimetric means, such as a group labeled with a fluorescent group, e.g. a group originating from a fluorescent dye. E.g. the group DYE is a group comprising a fluorescent group, such as a fluorescent group as conventional, e.g. including a pyrenyl, a dansyl, a nitrobenzo-2-oxa-1,3-diazolyl (=NBD), a Cy5 group, a bodipy group, such as a pyrenyl, a dansyl, a nitrobenzo-2-oxa-1,3-diazolyl (=NBD). The group DYE is preferably a
a pyrenyl,
a dansyl,
a nitrobenzo-2-oxa-1,3-diazolyl (=NBD-yl).

In another aspect the present invention provides a compound of formula I, wherein R₁, R₂, X, A-D, m, E-G, n, L-M are as defined above, and the group DYE is a pyrenyl or a dansyl.

The group DYE is bound chemically in a compound of formula I and is derived from a molecule having a group which may be selectively detected by physical means in its chemical structure. Said selective detectability remains in a compound of formula I. E.g. a molecule comprising a fluorescent group is bound chemically in a compound of formula I and is derived from a molecule having a fluorescent group in its chemical structure, which fluorescent group retains its fluorescent characteristics in a compound of formula I.

Pyrene is a compound of formula

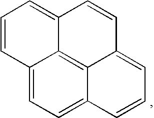

pyren-2-Yl is a group of formula

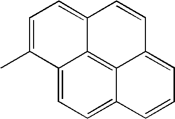

dansyl is a group of formula

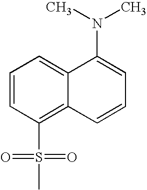

NBD is a group of formula

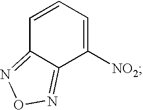

nitrobenzo-2-oxa-1,3-diazol-7-Yl is a group of formula

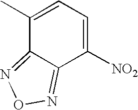

The basic chemical structure of the bodipy fluorophore is of formula

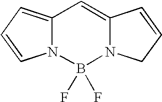

Cy5 includes the cyanine-5.18 molecule of formula

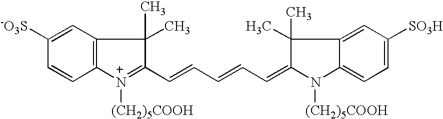

A pyrenyl, a dansyl, a nitrobenzo-2-oxa-1,3-diazolyl (=NBD), a Cy5 group and a bodipy group include fluorescent groups comprising the corresponding basic chemical structure as set out above and also includes fluorescent groups having chemical modifications of said structures.

In another aspect the present invention provides
a compound of formula

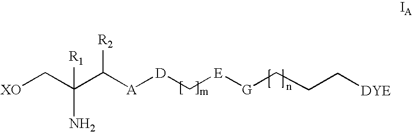

a compound of formula

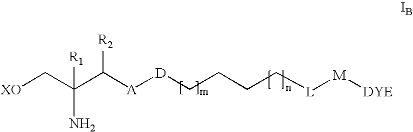

a compound of formula

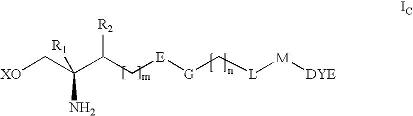

wherein
R₁, R₂, X, A-D, E-G, L-M, m, n and the group DYE are as defined above, with the proviso that
at least one of E-G and L-M is selected from the group consisting of CH₂—NH, CH₂—N((C₁₋₄)alkyl), CH₂—O, phenyl-O, O—CO, CO—O, CO—NH, NH—CO, CO—N((C₁₋₄)alkyl), N(C₁₋₄)alkyl)-CO, NH—SO₂, N((C₁₋₄)alkyl)-SO₂, and
a compound of formula

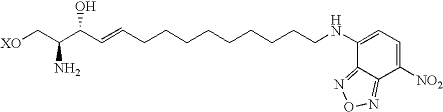

wherein X is as defined above, is excluded.

In another aspect the present invention provides
a compound of formula

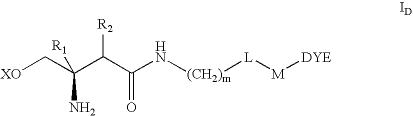

a compound of formula

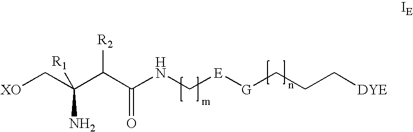

wherein

R₁, R₂, X, E-G, L-M, n, m and the group DYE are as defined above, with the proviso that at least one of E-G and L-M is selected from the group consisting of CH₂—NH, CH₂—N((C₁₋₄)alkyl), CH₂—O, phenyl-O, O—CO, CO—O, CO—NH, NH—CO, CO—N((C₁₋₄)alkyl), N(C₁₋₄)alkyl)-CO, NH—SO₂, N((C₁₋₄)alkyl)-SO₂.

A compound of formula I includes a compound of formula I_{A}, I_{B}, I_{C}, I_{D} and I_{E}.

A sphingosine of the present invention includes a compound of formula I (e.g. and a compound of formula I_{P1} and I_{P2}).

In a preferred aspect in a compound of formula I
R₁ is H or methyl,
R₂ is H or OH,
X is H or (HO)₂PO,
A-D is present and is CH═CH, CO—NH, O—CH₂ or a group of formula

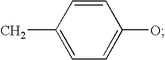

or A-D is absent,
E-G is O—CO, CH₂—CH₂, NH—CO, or a group of formula

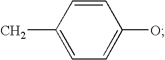

L-M is CH₂—CH₂, CH₂—NH, O—CH₂, O—CO, NH—CO, NH—SO₂,
m is a number selected from 0 to 12, e.g. 0 to 10,
n is a number selected from 0 to 12, e.g. 0 to 8,
m plus n is a number selected from 0 to 14, e.g. 0 to 11,
   the group DYE is a group selected from the group consisting of a pyrenyl, a dansyl and a nitrobenzo-2-oxa-1,3-diazol-yl.
   with the proviso that
at least one of E-G and L-M is selected from the group consisting of CH₂—NH, CH₂—N((C₁₋₄)alkyl), CH₂—O, phenyl-O, O—CO, CO—O, CO—NH, NH—CO, CO—N((C₁₋₄)alkyl), N(C₁₋₄)alkyl)-CO, NH—SO₂, N((C₁₋₄)alkyl)-SO₂, and
a compound of formula

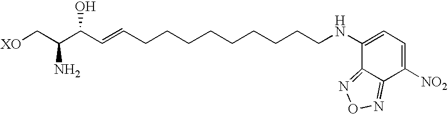

wherein X is as defined above, is excluded.

In a further aspect the present invention provides a compound of formula I, wherein

### R₁ is H,

### R₂ is H or OH,

X is H or (HO)₂PO, e.g. H,
A-D is present and is CH═CH, CO—NH or O—CH₂, or A-D is absent,
E-G is CH₂—CH₂, O—CO, NH—CO or a group of formula

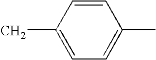

### L-M is CH₂—CH₂, O—CO, NH—CO, O—CH₂ or NH—SO₂,

m is a number selected from 0 to 10,
n is a number selected from 0 to 7,
m plus n is a number selected from 0 to 10, and
the group DYE is a pyrenyl;
with the proviso that, if L-M is CH₂—CH₂, than E-G is O—CO or NH—CO.

In a further aspect the present invention provides a compound of formula I, wherein

R₁ is H or methyl,

### R₂ is H or OH,

X is H or (HO)₂PO, e.g. H,

### A-D is CH═CH or O—CH₂,

### E-G is CH₂—CH₂,

### L-M is CH₂—NH,

m is a number selected from 0 to 7,
n is 0,
m plus n is a number selected from 5 to 10, e.g. 7, and
the group DYE is a dansyl.

In a further aspect the present invention provides a compound of formula I, wherein

R₁ is H or methyl,

### R₂ is H or OH,

### X is H or (HO)₂PO,

A-D is CH═CH, CO—NH, O—CH₂ or a group of formula

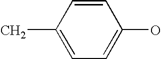

### E-G is CH₂—CH₂,

### L-M is CH₂—NH,

m is a number selected from 3, 4 5, 7 or 8,
n is a number selected from 0 or 1,
m plus n is a number selected from 3 to 8, and
the group DYE is nitrobenzo-2-oxa-1,3-diazol-yl.

In another aspect the present invention provides a compound of formula I, wherein X is H, unless given otherwise, and
R₁ is H, R₂ is OH, A-D is CH═CH, m is 3, E-G is O—CO, n is 1, L-M is CH₂—CH₂, and the group DYE is pyren-2-yl,
R₁ is H, R₂ is OH, A-D is CH═CH, m is 3, E-G is O—CO, n is 3, L-M is CH₂—CH₂, and the group DYE is pyren-2-yl,
R₁ is H, R₂ is OH, A-D is CH═CH, m is 3, E-G is O—CO, n is 7, L-M is CH₂—CH₂, and the group DYE is pyren-2-yl,
R₁ is H, R₂ is OH, A-D is CH═CH, E-G is CH₂—CH₂, m plus n are 8, L-M O—CO, and the group DYE is pyren-2-yl,
R₁ is H, R₂ is OH, A-D is CH═CH, E-G is CH₂—CH₂, m plus n are 8, L-M is NH—CO, and the group DYE is pyren-2-yl,
R₁ is H, R₂ is OH, A-D is CH═CH, E-G is CH₂—CH₂, m plus n are 8, L-M is NH—SO₂, and the group DYE is pyren-2-yl,
R₁ is H, R₂ is OH, A-D is CH═CH, E-G is CH₂—CH₂, m plus n are 7, L-M is CH₂—NH, and the group DYE is dansyl,
R₁ and R₂ are H, A-D is CO—NH, m is 6, E-G is O—CO, n is 1, L-M is CH₂—CH₂, and the group DYE is pyren-2-yl,
R₁ and R₂ are H, A-D is CO—NH, E-G is CH₂—CH₂, m plus n are 8, L-M is NH—CO, and the group DYE is pyren-2-yl,
R₁ and R₂ are H, A-D is CO—NH, E-G is CH₂—CH₂, m plus n are 8, L-M is NH—SO₂, and the group DYE is pyren-2-yl,
R₁ and R₂ are H, A-D is CO—NH, m is 10, E-G is NH—CO, n is 1, L-M is CH₂—CH₂, and the group DYE is pyren-2-yl,
R₁ and R₂ are H, A-D is CO—NH, E-G is CH₂—CH₂, m plus n are 7, L-M is CH₂—NH, and the group DYE is nitrobenzo-2-oxa-1,3-diazol-7-yl,
R₁ is methyl, R₂ is H, A-D is absent, E-G is a group of formula

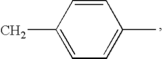

m and n are 0, L-M is O—CH₂, and the group DYE is pyren-2-yl,
R₁ and R₂ are H, A-D is O—CH₂, E-G is CH₂—CH₂, m plus n are 7, L-M is CH₂—NH, and the group DYE is nitrobenzo-2-oxa-1,3-diazol-7-yl,
R₁ is methyl, R₂ is H, A-D is O—CH₂, E-G is CH₂—CH₂, m plus n are 7, L-M is CH₂—NH, and the group DYE is nitrobenzo-2-oxa-1,3-diazol-7-yl,
R₁ is methyl, R₂ is H, A-D is O—CH₂, E-G is CH₂—CH₂, m plus n are 7, L-M is CH₂—NH, and the group DYE is dansyl,
R₁ is methyl, R₂ is H, A-D is O—CH₂, E-G is CH₂—CH₂, m plus n are 8, L-M is NH—CO, and the group DYE is pyren-2-yl,
R₁ and R₂ are H, A-D is O—CH₂, E-G is CH₂—CH₂, m plus n are 8, L-M is NH—CO, and the group DYE is pyren-2-yl,
R₁ is methyl, R₂ is H, A-D is a group of formula

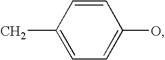

E-G is CH₂—CH₂, m plus n are 5, L-M is CH₂—NH, and the group DYE is nitrobenzo-2-oxa-1,3-diazol-7-yl,
R₁ is methyl, R₂ is H, A-D is a group of formula

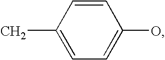

E-G is CH₂—CH₂, m plus n are 8, L-M is CH₂—NH, and the group DYE is nitrobenzo-2-oxa-1,3-diazol-7-yl,
R₁ is methyl, R₂ is H, A-D is a group of formula

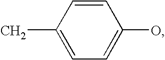

E-G is CH₂—CH₂, m plus n are 3, L-M is CH₂—NH, and the group DYE is nitrobenzo-2-oxa-1,3-diazol-7-yl,
R₁ is methyl, R₂ is H, X is (OH)₂PO, A-D is a group of formula

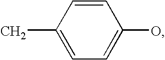

E-G is CH₂—CH₂, m plus n are 8, L-M is CH₂—NH, and the group DYE is nitrobenzo-2-oxa-1,3-diazol-7-yl,
R₁ and R₂ are H, X is (OH)₂PO, A-D is O—CH₂, E-G is CH₂—CH₂, m plus n are 7, L-M is CH₂—NH, and the group DYE is nitrobenzo-2-oxa-1,3-diazol-7-yl,
R₁ is H, R₂ is OH, A-D is CH═CH, E-G is CH₂—CH₂, m plus n are 7, L-M is CH₂—NH, and the group DYE is nitrobenzo-2-oxa-1,3-diazol-7-yl,
R₁ is H, R₂ is OH, X is (OH)₂PO, A-D is CH═CH, E-G is CH₂—CH₂, m plus n are 7, L-M is CH₂—NH, and the group DYE is nitrobenzo-2-oxa-1,3-diazol-7-yl.

Compounds provided by the present invention are hereinafter designated as “compound(s) of (according to) the present invention”. A compound of formula I includes a compound of formula I_{A}, I_{B}, I_{C}, I_{D} and I_{E}. A labeled sphingosine, e.g. a compound of the present invention includes a compound in any form, e.g. in free form, in the form of a salt, in the form of a solvate and in the form of a salt, optionally in the form of a solvate where existing.

In another aspect the present invention provides a compound of formula I, wherein R₁, R₂, X, A-D, m, E-G, n, L-M and the group DYE are as defined above, with both provisos of above, in the form of a salt.

A salt of labeled sphingosines, e.g. a compound of the present invention, includes a metal salt and an acid addition salt. Metal salts include for example alkali or earth alkali salts; e.g. potassium, sodium; acid addition salts include salts of a compound of formula I with an acid, e.g. hydrogen fumaric acid, fumaric acid, naphthalin-1,5-sulphonic acid, hydrochloric acid, deuterochloric acid; preferably hydrochloric acid.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of isomers and mixtures thereof; e.g. optical isomers, diastereoisomers, cis/trans conformers. A compound of the present invention may e.g. contain asymmetric carbon atoms and may thus exist in the form of enantiomers or diastereoisomers and mixtures thereof, e.g. racemates. Any asymmetric carbon atom may be present in the (R)-, (S)- or (R,S)-configuration, preferably in the (R)- or (S)-configuration. For example, in a compound of the present invention the carbon atom to which R₁ is attached and the carbon atom to which R₂ is attached (if R₂ is other than hydrogen), both are asymmetric carbon atoms; groups attached to asymmetric carbon atoms may be in the (R)-, (S)- or (R,S)-configuration. E.g. a compound of the present invention may comprise a double bond and hydrogen atoms or groups at such double bond may thus be in the cis- or in the trans conformation.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula I, where tautomers can exist.

In a compound of formula I, I_{A}, I_{B}, I_{C}, I_{D} and I_{E} each single defined substitutent may be a preferred substituent, e.g. independently of each other substitutent defined.

A phosphate source in a method provided by the present invention includes a phosphate source present in natural environment, such as ATP or GTP, preferably ATP.

A sample in a method comprising steps a. to c., or, A to D, respectively, as provided by the present invention includes any sample in which activity of a sphingosine kinase or a sphingosine phopshate phosphatase is desired to be determined, e.g. including samples from in vivo and in vitro investigations, e.g. samples comprising cells, e.g. including cell lysates, tissue, or serum from yeast, mammals, animals, birds, fish and plants; in vitro samples, test samples comprising no kinase activity, e.g. for comparison reasons, in an appropriate purity. For determination of a sphingosine phosphate phosphatase in vivo samples are used. Provision of such samples may be carried out as appropriate, e.g. according, e.g. analogously, to a method as conventional.

In case no enzyme activity is present in a sample, no activity will be detected when a method of the present invention is used. In case that kinase/phosphatase activity is comprised in the sample, the extent of the activity may be determined by use of a method of the present invention.

Sphingosine kinase activity in a method of the present invention includes the activity of any sphingosine kinase, e.g. of sphingosine kinase-1 and of sphingosine kinase-2. Sphingosine phosphate phosphatase activity in a method of the present invention includes the activity of any sphingosine phosphatase involved in the sphingolipid pathway, such as e.g. a sphingosine phosphate phosphatase and a lipid phosphate phosphatase.

The period of time necessary for the formation of an enzymatic product in a method provided by the present invention depends on the results desired in investigation. Such time period may be predetermined in test samples for each individual sample.

The enzymatic product formed In a method provided by the present invention is either a phosphate containing enzymatic, labeled product, in case of a shingosine kinase, such as a phosphorylated fluorescent labeled sphingosine, e.g. a compound of formula I, wherein the residues are as defined above and wherein the hydroxy group in position 1 of the corresponding sphingosine is phosphorylated, i.e. the hydroxy group in position 1 is replaced by the (HO)₂PO—O— group; or an enzymatic labeled unphosphorylated product in case of a sphingosine phosphate ühosphatase, e.g. a compound of formula I, wherein the residues are as defined above and wherein the hydroxy group in position 1 of the corresponding sphingosine is free and unphosphorylated.

Organic solvent which is able to form two phases in combination with water in a method provided by the present invention is able
to form two phases in combination with water, and
to dissolve unreacted labeled sphingosine in a mixture of said organic solvent and water.

Such organic solvent includes e.g. halogenated hydrocarbons, such as CHCl₂, CHCl₃, ethers, such as tetrahydrofurane, diethylether; hydrocarbons, such as n-butane, n-pentane; alkyl acetates, e.g. (C₂₋₅)alkylacetate, such as ethyl acetate, propyl acetates, butyl acetates, e.g. including mixtures of individual organic solvents, e.g. such as specified herein.

An aqueous buffer solution in a method provided by the present invention is a buffer solution having a pH at which an enzymatic product formed is able to dissolve in the aqueous phase in a mixture of organic solvent as defined above and aqueous buffer solution, e.g. an pH of 7.0 to 10, such as, e.g. around, 8.5, e.g. including an K₂HPO₄ containing buffer. Such aqueous buffer solutions are known or may be obtained as conventional.

The separation of the two phases formed in a method provided by the present invention may be carried out as appropriate, e.g. according, e.g. analogously, to a method as conventional, e.g. by extraction of the aqueous phase obtained in step b, or step B., respectively, with organic solvent and separating the phases obtained in step c or step C., respectively.

The amount of enzymatic product formed in a method provided by the present invention may be determined as appropriate, e.g. according, e.g. analogously, to a method as conventional, e.g. by fluorimetric means at appropriate wave lengths, e.g. in the aqueous phase obtained after phase separation.

Methods according to the present invention may be useful for the identification of an agent that modulates the activity of a sphingosine kinase, or a phosphatase involved in the sphingolipid pathway, respectively.

In another aspect the present invention provides a method for identifying an agent that modulates the activity of a sphingosine kinase comprising the steps of
a. contacting a labeled unphosphorylated sphingosine with
   a phosphate source, and
   a sphingosine kinase,
   for a predetermined period of time so that an enzymatic product can be formed,
a1. in the absence of a candidate compound, and
a2. in the presence of a candidate compound,
b. adding to the mixture of step a1 and of step a2 an aqueous buffer solution and organic solvent which is able to form two phases in combination with water,
c. separating the unreacted labeled sphingosine from the enzymatic product formed in steps a1. and a2., e.g. according to method steps b. and c. as defined in a method according to the present invention,
d. detecting the amount of enzymatic product obtained in step a1. and in step a2., e.g. by fluorimetric means, and
   determining whether there is a difference in the amount of enzymatic products formed in step a1. and step a2.,
e. choosing an agent that modulates the activity of a kinase as determined in step d.

In another aspect the present invention provides a method for identifying an agent that modulates the activity of a phosphatase involved in the sphingolipid pathway comprising the steps of
A. contacting a labeled phosphorylated sphingosine with living cells comprised in an appropriate medium for a predetermined period of time so that an enzymatic product can be formed,
A1. in the absence of a candidate compound, and
A2. in the presence of a candidate compound,
B. separating the unreacted labeled sphingosine from the enzymatic product formed in steps A1. and A2.,
C. detecting the amount of enzymatic product obtained in step A1. and in step A2 and determining whether there is a difference in the amount of enzymatic products formed in step A1. and step A2.,
D. choosing an agent that modulates the activity of a phosphatase involved in the sphingolipid pathway as determined in step C.

An agent identified by methods provided by the present invention is herein also designated as “An agent of the present invention”.

A method provided by the present invention using a labeled, e.g. a fluorescent labeled, sphingosine may be a useful tool for the determination of activity of an enzyme selected from the group consisting of a sphingosine kinase and a phosphatase involved in the sphingolipid pathway, such as e.g. a sphingosine phosphate phosphatase and a lipid phosphate phosphatase, and in the screening for pharmaceutically active compounds which mediate (influence) the activity of a sphingosine kinase. Appropriate TEST systems for assaying an enzyme activity selected from the group consisting of a sphingolipid kinase and a phosphatase involved in the sphingolipid pathway, such as e.g. a sphingolipid phosphate phosphatase and a lipid phosphatase, are described in the examples.

It is an advantage that a method provided by the present invention may be carried out in high-throughput screening (HTS).

A candidate compound includes compound (libraries) from which the effect on an enzyme selected form the group consisting of a sphingolipid kinase and a phosphatase involved in the sphingolipid pathway, such as e.g. a sphingolipid phosphate phosphatase and a lipid phosphate phosphatase, are unknown. Compound (libraries) include for example oligopeptides, polypeptides, proteins, antibodies, mimetics, small molecules, e.g. low molecular weight compounds (LMW's).

An agent of the present invention is a candidate compound from which an effect on the activity of an enzyme selected form the group consisting of a sphingolipid kinase and a phosphatase involved in the sphingolipid pathway, such as e.g. a sphingolipid phosphate phosphatase and a lipid phosphatase, has been determined in a method provided by the present invention. Such agent may decrease or enhance the activity of a kinase/phosphatase. An agent of the present includes oligopeptides, polypeptides, proteins, antibodies, mimetics, small molecules, e.g. low molecular weight compounds (LMW's).

In another aspect the present invention provides a kit for determining the activity of an enzyme selected from the group consisting of a sphingosine kinase and a phosphatase involved in the sphingolipid pathway, such as e.g. a sphingosine phosphate phosphatase and a lipid phosphate phosphatase comprising as a main component a labeled sphingosine, e.g. a fluoresent labeled sphingosine, e.g. a compound of the present invention, such as a compound of formula I (e.g. or of formulae I_{P1} or I_{P2}) as described herein, and instructions for using said kit, e.g. comprising an indication, e.g. detailed, for carrying out step a. to c. and/or steps A. to D. as defined in one of the methods of the present invention.

Such kit may further comprise a substantial component including an appropriate environment of a sample to be tested.

Such kit may be useful for identifying an agent that mediates the activity of an enzyme selected from the group consisting of a sphingosine kinase and a phosphatase involved in the sphingolipid pathway, such as e.g. a sphingosine phosphate phosphatase and a lipid phosphate phosphatase.

For determining the activity of a kinase a labeled unphosphorylated sphingosine is used, for determining of a the activity of a phosphatase involved in the sphingolipid pathway a labeled 1-phosphorlyated sphingosine is used in a kit of the present invention.

In another aspect the present invention provides a kit as described above for use in the identification of an agent that mediates the activity of an enzyme selected from the group consisting of a sphingosine kinase and a phosphatase involved in the sphingolipid pathway, such as e.g. a sphingosine phosphate phosphatase and a lipid phosphate phosphatase, e.g. which kit may further comprise as a substantial component e.g. appropriate means and indications to determine the effect of a candidate compound in a sample to be tested.

Furthermore we have found surprisingly that the phosphate conversion rate of sphingosine kinase-1- or sphingosine kinase-2 in a method provided by the present invention may be different, maybe due to the use of a sphingosine of the present invention. In a method provided by the present invention it may be thus possible to distinguish whether in a sample sphingosine kinase-1- or sphingosine kinase-2 (or no sphingosine kinase activity) is present.

In another aspect the present invention provides a method for determining whether in a sample sphingosine kinase-1-activity, or sphingosine kinase-2-activity, or both, or no sphingosine kinase activity, is present, comprising the steps of
α. contacting
α1. a labeled unphosphorylated sphingosine with a sample which sample optionally comprises sphingosine kinase-1-activity, or sphingosine kinase-2-activity, or both, or no sphingosine kinase activity, with a phosphate source,
α2. a labeled unphosphorylated sphingosine with a sample comprising a defined amount of sphingosine kinase-1-activity with a phosphate source,
α3. a labeled unphosphorylated sphingosine with a sample comprising a defined amount of sphingosine kinase-2-activity with a phosphate source, for a predetermined period of time, so that an enzymatic product can be formed,
β. separating the unreacted labeled sphingosine from the enzymatic product formed in steps α1., α2. and α3., e.g. according to method steps b. and c. as defined in a method provided by the present invention, and
γ. determining and comparing the phosphate conversion rate in steps a1., a2. and a3.

In a further aspect the present invention provides a method for differentiating whether a test compound is capable to mediate the activity of a sphingosine kinase-1 and/or a sphingosine kinase-2 comprising the steps
i. contacting a labeled unphsophorylated sphingosine with a phosphate source and with
i1. a sphingosine kinase-1,
i2. a sphingosine kinase-2,
   in the absence of a test compound, and
   in the presence of a test compound
   for a predetermined period of time so that an enzymatic product can be formed,
ii. separating the unreacted labeled sphingosine from the enzymatic product formed in steps i1. and i2., e.g. according to method steps b. and c. as defined in a method provided by the present invention,
iii. determining and comparing the phosphate conversion rate in steps i1. and i2.

By determination of the phosphate conversion rate and comparison it may be possible to identify, whether a test compound is capable to mediate the activity of a sphingosine kinase-1 or of a sphingosine kinase-2, or both, e.g. and to which extent.

A test compound includes a candidate compound as defined herein, a compound from which it is known that it is able to mediate a kinase activity and an agent of the present invention.

In a method provided by the present invention comparative test samples are run in parallel under the same conditions (e.g. containing no sphingosine kinase activity).

In another aspect the present invention provides a process for the production of a compound of formula I (e.g. or of formula I_{P1} or I_{P2}) comprising the steps

a1. reacting a compound of formula

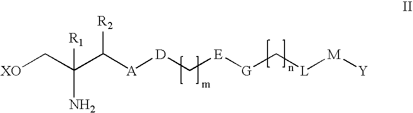

wherein R₁, R₂, X, A-D, E-G, L-M, n and m are as defined above and wherein Y is a group prone to substitute the leaving group Z in a compound of formula

DYE-Z III
with a compound of formula III, wherein the group DYE is as defined above, e.g. DYE-Z is a leaving group prone to substitute the group Y in a compound of formula II, and wherein functional groups are optionally protected, or
   a2. reacting a compound of formula

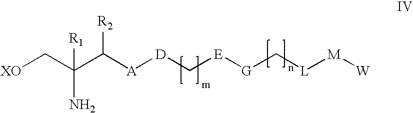

wherein R₁, R₂, X, A-D, E-G, L-M, n and m are as defined above and W is a leaving group, and wherein functional groups are optionally protected,
with a compound of formula

DYE-U V
wherein DYE is as defined above, U is a group prone to substitute the leaving group W, e.g. DYE-U is a group prone to substitute the leaving group W, and wherein functional groups are optionally protected,
to obtain a compound of formula I, wherein functional groups are optionally protected, or
   b. reacting a compound of formula

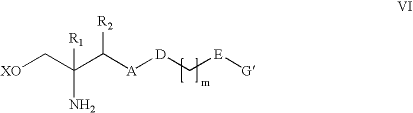

wherein R₁, R₂, X, A-D and m are as defined above, E-G′ has the meaning of E-G as defined above, but wherein G in E-G is replaced by G′, G′ is amino, (C₁₋₄)alkylamino hydroxy, carboxyl, a reactive carboxyl derivative, e.g. carboxylic acid halogenide, amide; or SO₃H, and wherein functional groups are optionally protected,
with a compound of formula

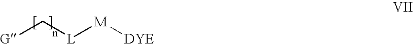

wherein L, M and DYE are as defined above and G″ is a group which is prone to react with G′ to obtain a group E-G in a compound of formula I obtained, wherein E-G is as defined above,
   c. optionally deprotecting a protected compound of formula I obtained, and
   d. isolating a compound of formula I from the reaction mixture.

A compound of formula I thus obtained may be converted into another compound of formula I, e.g. or a compound of formula I obtained in free form may be converted into a salt of a compound of formula I and vice versa.

The above reaction in steps a1., a2. and b. are nucleophilic substitution-reactions and may be carried out as appropriate, e.g. according, e.g. analogously, to a method as conventional, or as described herein.

Compounds of formula II, III, IV, V, VI and VII are intermediates (starting materials) in the preparation of compounds of formula I. Any compound described herein, e.g. a compound of the present invention and intermediates of formula II, III, IV, V, VI and VII may be prepared as appropriate, e.g. according, e.g. analogously, to a method as conventional, e.g. or as specified herein.

A typical process for the production of an Intermediate, such as a compound of formula IV, may be carried out according, e.g. analogously, to the following reaction SCHEME 1:

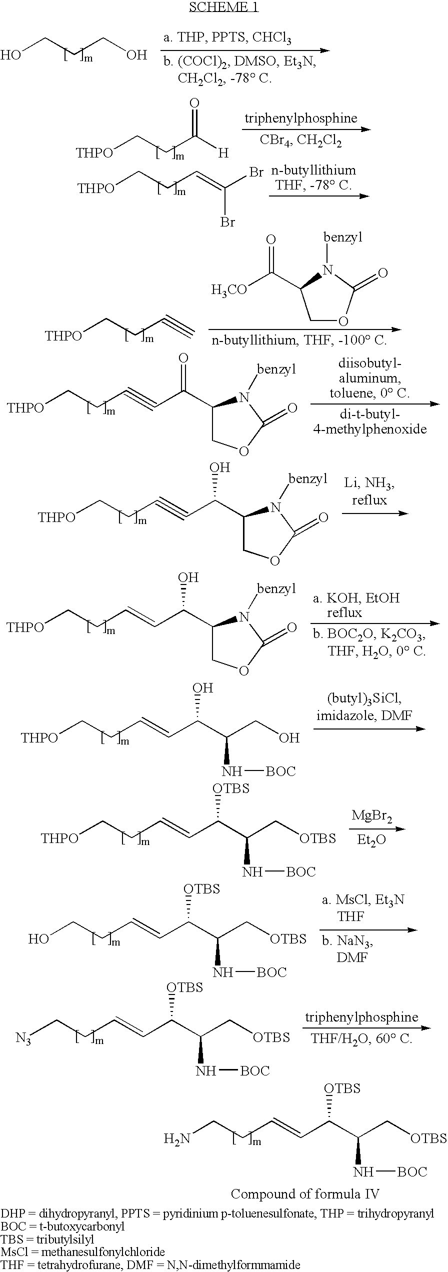

Another typical process for the production of an intermediate, e.g. for the production of a compound of formula VI, may be carried out according, e.g. analogously, to the following reaction SCHEME 2. In reaction SCHEME 2 also the production of a compound of formula I is shown:

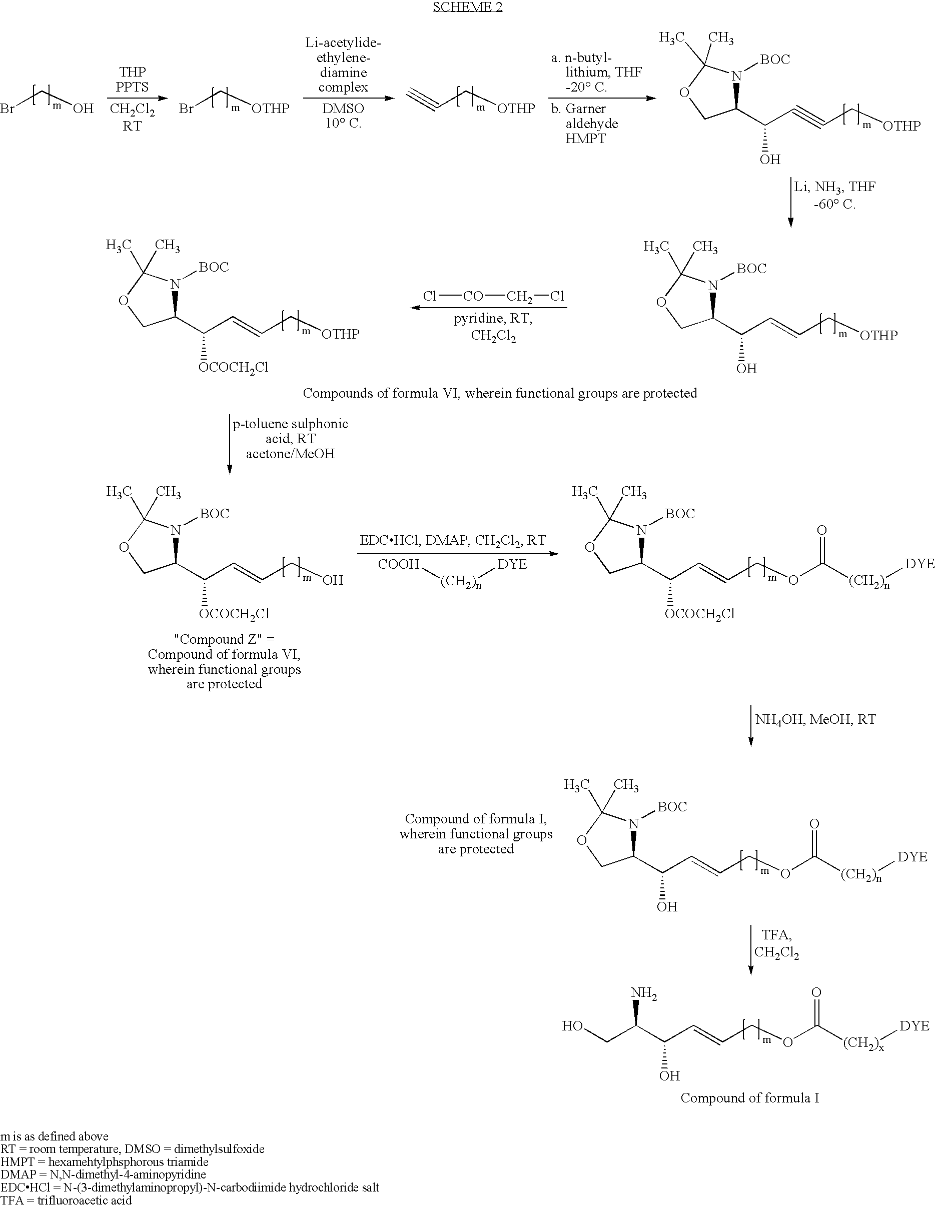

Another typical process for the production of an intermediate may be carried out according, e.g. analogously, to the following reaction SCHEME 3A or 3B.

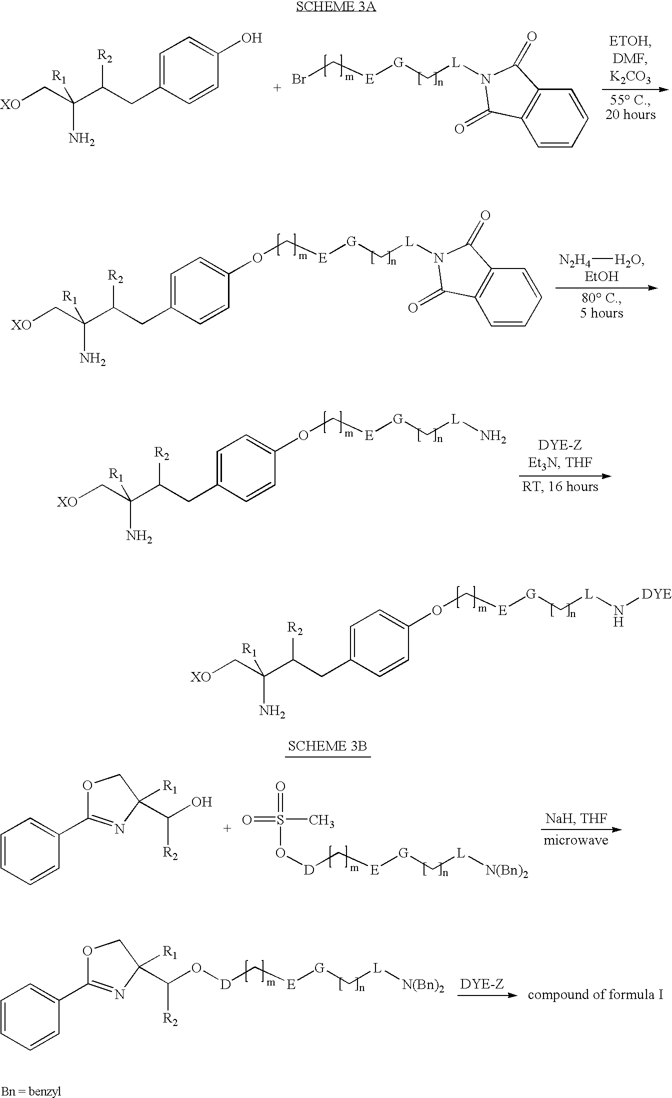

In SCHEME 1, SCHEME 2 and SCHEMEs 3A and 3B also appropriate protecting group technology is exemplified; e.g. functional groups in DYE may additionally be protected.

In another aspect the present invention provides the use of a labeled sphingosine, e.g. fluorescent labeled, of the present invention in a high-throughput assay.

In another aspect the present invention provides the use of a fluorescent labeled sphingosine of the present invention for the identification of an agent that mediates the activity of an enzyme selected from the group consisting of a sphingosine kinase and a and a phosphatase involved in the sphingolipid pathway, such as e.g. a sphingosine phosphate phosphatase and a lipid phosphatase, e.g. in a high-throughput assay.

In another aspect the present invention provides the use of a compound of the present invention for determining whether an agent is capable to mediate the activity of a sphingosine kinase, sphingosine kinase-1, or a sphingosine kinase-2, or both.

An agent provided by a method of the present invention may mediate the activity of an enzyme selected from the group consisting of a sphingosine kinase and a phosphatase involved in the sphingolipid pathway, such as e.g. a sphingosine phosphate phosphatase and a lipid phosphatase, and may thus exhibit pharmacological activity and may be useful as a pharmaceutical. An agent provided by a method of the present invention may show therapeutic activity against disorders mediated by an enzyme selected from the group consisting of a sphingosine kinase and a phosphatase involved in the sphingolipid pathway, such as e.g. a sphingosine phosphate phosphatase and a lipid phosphatase.

In another aspect the present invention provides an agent which is capable to modulate, e.g. inhibit or enhance, activity of an enzyme selected from the group consisting of a sphinosine kinase and a phosphatase involved in the sphingolipid pathway, such as e.g. a sphingosine phosphate phosphatase and a lipid phosphatase, which agent is identified by a method of the present invention, e.g. for use as a pharmaceutical.

Such disorders include diseases wherein the biological functions of SP, SPP, SPHKs or phosphatases involved in the sphingolipid pathway play a role. Such disorders e.g. include autoimmune disorders and inflammatory disorders, allergic disorders and cancer. Allergic disorders include e.g. allergic asthma, contact allergy, drug allergy, food allergy, atopic dermatitis or seasonal allergies such as allergic rhinitis.

Autoimmune related and inflammatory disorders include e.g. type I diabetes, multiple sclerosis, rheumatoid arthritis, psoriasis, inflammatory bowel disease, atherosclerosis or systemic Lupus erythematosus.

For pharmaceutical use an agent of the present invention for treatment includes one or more, preferably one, agent of the present invention, e.g. a combination of two or more agents of the present invention.

In a further aspect the present invention provides a pharmaceutical composition comprising an agent of the present invention as an active ingredient in association with at least one pharmaceutical excipient.

In another aspect the present invention provides the use of an agent of the present invention for the manufacture of a medicament, e.g. a pharmaceutical composition, for the treatment of disorders where the biological functions of SP, SPP, SPHKs or phosphatases involved in the sphingolipid pathway play a role, including autoimmune disorders and inflammatory disorders, allergic disorders and cancer, such as allergic asthma, contact allergy, drug allergy, food allergy, atopic dermatitis or seasonal allergies. e.g. allergic rhinitis; type I diabetes, multiple sclerosis, rheumatoid arthritis, psoriasis, inflammatory bowel disease, atherosclerosis, systemic Lupus erythematosus, cancer.

Treatment includes treatment and prophylaxis.

For such treatment, the appropriate dosage will, of course, vary depending upon, for example, the chemical nature and the pharmacokinetic data of an agent of the present invention employed, the individual host, the mode of administration and the nature and severity of the conditions being treated. However, in general, for satisfactory results in larger mammals, for example humans, an indicated daily dosage is in the range from about 0.01 g to about 2.0 g, of an agent of the present invention; such as 0.5 g to 1.0 g, conveniently administered, for example, in divided doses up to four times a day.

An agent of the present invention may be administered by any conventional route, for example enterally, e.g. including nasal, buccal, rectal, oral administration; parenterally, e.g. including intravenous, intramuscular, subcutaneous administration; or topically; e.g. including epicutaneous, intranasal, intratracheal administration;

e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

An agent of the present invention may be administered in the form of a pharmaceutically acceptable salt, e.g. an acid addition salt or metal salt; or in free form; optionally in the form of a solvate. An agent of the present invention in the form of a salt and optionally in the form of a solvate exhibits the same order of activity as an agent of the present invention in free form; optionally in the form of a solvate.

An agent of the present invention may be used for pharmaceutical treatment according to the present invention alone, or in combination with one or more other pharmaceutically active agents.

Combinations include fixed combinations, in which two or more pharmaceutically active agents are in the same formulation; kits, in which two or more pharmaceutically active agents in separate formulations are sold in the same package, e.g. with instruction for co-administration; and free combinations in which the pharmaceutically active agents are packaged separately, but instruction for simultaneous or sequential administration are given.

In another aspect the present invention provides a pharmaceutical composition comprising an agent of the present invention in association with at least one pharmaceutical excipient, e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides a pharmaceutical composition according to the present invention, further comprising another pharmaceutically active agent.

Such pharmaceutical compositions may be manufactured according, e.g. analogously, to a method as conventional, e.g. by mixing, granulating, coating, dissolving or lyophilizing processes. Unit dosage forms may contain, for example, from about 0.5 mg to about 2000 mg, such as 1 mg to about 500 mg, e.g. 0.00625 mg/kg to about 12.5 mg/kg.

In another aspect the present invention provides a method for determining the activity of a sphingosine kinase in a sample, comprising the steps of

a. contacting a labeled sphingosine with
a phosphate source, and
a sample which may comprise a sphingosine kinase,
for a predetermined period of time, so that an enzymatic product can be formed,
   b. adding to the mixture of step a. an aqueous buffer solution and organic solvent which is able to form two phases in combination with water,
   c. separating the phases obtained in step b., e.g. after extraction, and
   d. determining the amount of enzymatic product in the aqueous phase obtained in step c; e.g. wherein the labeled sphingosine is a compound of formula

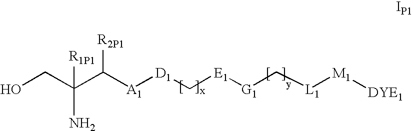

wherein
R_{1P1} is H or (C₁₋₄)alkyl, e.g. methyl, R_{2P1} is H, OH or oxo,
A₁, D₁, E₁, G₁, L₁ and M₁ independently of each other are CH₂, and additionally
A₁, D₁, E₁, G₁, L₁ and M₁ are independently of each other O, CO, NH, N((C₁₋₄)alkyl), e.g. N(CH₃), SO₂ or CH₂, with the proviso that compounds are excluded wherein in a group A₁-D₁, E₁-G₁ and/or L₁-M₁ the components A₁ and D₁, E₁ and G₁ and/or L₁ and M₁ are identical, or
A₁ and D₁, E₁ and G₁ and/or L₁ and M₁ together are a group CH═CH, and L₁ and M₁ together additionally are a group HC≡CH,
x is a number selected from 0 to 12,
y is a number selected from 0 to 12,
with the proviso that x plus y is a number selected from 3 to 14, and the group DYE₁ is a group selectively detectable in a compound of formula I_{P1} by physical means.

For studies on the function and subcellular localization of ceramide and for the identification of inhibitors of a ceramide kinase, which ceramide kinases are also involved in the sphingolipid pathway, the determination of the bioactivity of a ceramide kinase may be essential. For such evaluations a labeled ceramide may be used, such as a labeled ceramide which originates from a labeled sphingosine according to the present invention which labeled sphingosine is unphosphorylated.

In another aspect the present provides the use of a labeled ceramide, which ceramide is originating from a sphingosine of formula I, wherein the residues are as defined above and wherein X is H, with both provisos as defined above,

e.g. which labeled ceramide may be formed during the sphingolipid pathway if a labeled sphingosine of formula I, wherein the residues are as defined above, and wherein X is H, with both provisos as defined above, is used as a starting material in such sphingolipid pathway,
for determining whether an activity of a ceramide kinase involved in the sphingolipid pathway is present in a sample or not, or determining the extent of said activity;
e.g. by a method of
a. contacting living cells comprised in an appropriate culture medium with a labeled ceramide originating from a labeled sphingosine of formula I wherein the residues are as defined above and wherein X is H, with both provisos of above, for a predetermined period of time so that an enzymatic product can be formed,
b. separating the enzymatic product formed in step a., and
c. determining the amount of enzymatic product formed.

Such method is also useful in a method for the identification of an agent which mediates the activity of a ceramide kinase involved in the sphingolipid pathway, which method comprises steps as described herein for the identification of an agent which mediates the activity of a sphingosine kinase or of a phosphatase involved in the sphingolipid pathway, but using a ceramide kinase instead of a sphingosine kinase or a phosphatase and instead of a labeled sphingosine of the present invention a labeled ceramide originating from a labeled sphingosine of formula I wherein the residues are as defined above and wherein X is H, e.g. which labeled ceramide may be formed during the sphingolipid pathway if a labeled sphingosine of formula I, wherein the residues are as defined above, and wherein X is H, with both provisos as defined above, is used as a starting material in such sphingolipid pathway.

Methods for determining enzyme activity in living cells according to the present invention may also be used for the determination of sphingosine kinase activity and phosphatase activity,

e.g. and of ceramide kinase activity,
in one single sample, if living cells are used in said sample, e.g. in which sample the sphingolipid pathway is occurring.

### DESCRIPTION OF THE FIGURES

FIG. 1 shows the distribution pattern given as results of densitometric measurements of sphingoid base intermediates when HUVECs are incubated with
a) labeled sphingosine (NBD-Sph) and the distribution pattern for the kinase activity is determined, or
b) labeled sphingosine-1 phosphate and the distribution pattern for the phosphatase activities are determined.
   The metabolite products are sphingosine (Sph), sphingosine-1-phopshate (S1P), ceramide (Cer) and sphingomyelin (SM).

FIG. 2 shows the intracellular conversion of labeled sphingosine of Example 24 in the presence of an SPHK-1 inhibitor.

FIG. 3 shows that a labeled sphingosine of Example 24 is phosphorylated more efficient by SPHK-2 than by SPHK-1 when contacted with appropriate cells.
lane 1: pattern in case cells are transfected with a control vector only,
lane 2: pattern in case cells overexpress SPHK-1
lane 3: pattern in case cells overexpress SPHK-2.

In the following examples all temperatures are in degree centigrade (° C.) and are uncorrected.

The following ABBREVIATIONS are used

ACN acetonitrile
AcOH acetic acid
aq. aqueous
BuOH butanol
DMAP N,N-dimethyl-4-aminopyridin
DMF dimethylformamide
DMSO dimethylsulfoxide
D_{D} dansyl

### EtAc Ethylacetate

MeOH methanol
N_{D} nitrobenzo-2-oxa-1,3-diazol-7-yl (NBD)
P_{D} pyren-2-yl
RT room temperature
sat. saturated
SPK-1, SPK-2 sphingosine kinase-1, shingosine kinase-2
S1P, SPP sphingosine 1 phosphate
SM sphingomyelin
TLC thin layer chromatography
TFA trifluoroacetic acid

### EXAMPLE A

### Pyrene-1-carboxylic acid (E)-(13R,14S)-14-amino-13,15-dihydroxy-pentadec-11-enyl) ester

A solution of 508 mg of (S)-4-[(E)-(R)-1-hydroxy-13-(pyrene-1-carbonyloxy)-tridec-2-enyl]-2,2-dimethyl-oxazolidine-3-carboxylic acid tert-butyl ester in 15 ml of CH₂Cl₂ is treated with 1 ml of TFA and stirred at RT overnight. From the mixture obtained solvent is evaporated, the evaporation residue is subjected to preparative RP-chromatography and pyrene-1-carboxylic acid (E)-(13R,14S)-14-amino-13,15-dihydroxy-pentadec-11-enyl ester is obtained.

### Production of STARTING MATERIALS

### A1. (S)-4-[(E)-(R)-1-Hydroxy-13-(pyrene-1-carbonyloxy)-tridec-2-enyl]-2,2-dimethyl-oxazolidine-3-carboxylic acid tert-butyl ester

A solution of 580 mg of (S)-4-[(E)-(R)-1-(2-chloro-acetoxy)-13-(pyrene-1-carbonyloxy)-tridec-2-enyl]-2,2-dimethyl-oxazolidine-3-carboxylic acid tert-butyl ester in 15 ml of MeOH and 2 ml of CH₂Cl₂ is treated with 5 ml of aqueous NH₄OH (28% w/w) and stirred for 5 hours at RT. From the reaction mixture obtained solvent is evaporated and the evaporation residue obtained is distributed between 1N aqueous HCl and EtAc. The organic layer obtained is washed with brine, dried and solvent is evaporated.

(S)-4-[(E)-(R)-1-Hydroxy-13-(pyrene-1-carbonyloxy)-tridec-2-enyl]-2,2-dimethyl-oxazolidine-3-carboxylic acid tert-butyl ester is obtained.

### A2. (S)-4-[(E)-(R)-1-(2-Chloro-acetoxy)-13-(pyrene-1-carbonyloxy)-tridec-2-enyl]-2,2-dimethyl-oxazolidine-3-carboxylic acid tert-butyl ester

A solution of 540 mg of (S)-4-[(E)-(R)-1-(2-chloro-acetoxy)-13-hydroxy-tridec-2-enyl]-2,2-dimethyl-oxazolidine-3-carboxylic acid tert-butyl ester (prepared analogously as described in: Kozikowski et al., Tetrahedron Lett. 1996, 37, 3279-3282) in 10 ml of dry CH₂Cl₂ is treated with 271 mg of pyrene-1-carboxylic acid, 422.4 mg of 1-(3-dimethylaminopropyl)-3-ethyl-carbodiimide HCl and 269.3 mg of DMAP and stirred for 4 hours at RT. The reaction mixture obtained is distributed between EtAc and 1 N aq. HCl. The organic layer obtained is washed with sat. aq. NaHCO₃ solution and brine, dried and solvent is evaporated.

(S)-4-[(E)-(R)-1-(2-Chloro-acetoxy)-13-(pyrene-1-carbonyloxy)-tridec-2-enyl]-2,2-dimethyl-oxazolidine-3-carboxylic acid tert-butyl ester is obtained.

### EXAMPLE B

### 4-Hydroxy-3-methyl-N-[10-(pyrene-1-sulfonylamino)-decyl]butyramide

4-Hydroxy-3-methyl-N-[10-(-1-sulfonylamino)-decyl]butyramide is prepared by condensation of O,N-protected (R)-3-amino-4-hydroxybutanoic acid and 1,10-diaminodecane. 25 mg of condensation product obtained are dissolved in dry CH₂Cl₂ and treated with 21 μl of Et₃N, catalytic amounts of DMAP and 15 mg of 1-pyrenesulfonyl chloride. The mixture obtained is stirred overnight at RT under protection from light. The mixture obtained is washed with H₂O, dried and solvent is evaporated. The evaporation residue obtained is filtrated under light protection and the filtration residue obtained is dissolved in MeOH/HCl solution and left for 30 minutes at RT. From the mixture obtained solvent is evaporated and 4-hydroxy-3-methyl-N-[10-(pyrene-1-sulfonylamino)-decyl]butyramide in the form of a hydrochloride is obtained.

Analogously to a method as described in Example A or B, but using appropriate starting materials, compounds of Examples 1 to 13 as set out in the following TABLES 1 and 2 are obtained. ¹H-NMR data (CDCl₃, 400 MHz, unless given otherwise) (or Electrospray MS data) also is indicated in TABLES 1 and 2.

### EXAMPLES 1 to 7

### Compounds of Formula

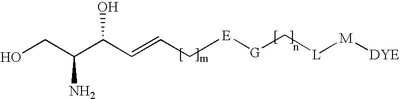

wherein m, E-G, L-M, n, m and DYE are as defined in TABLE 1:

TABLE 1

| | | | | | | ¹H-NMR data (DMSO-d₆, 500 MHz, |
|---|---|---|---|---|---|---|
| EX | E-G | L-M | m | n | DYE | unless given otherwise) or MS data |
| 1 | O—CO | CH₂—CH₂ | 3 | 1 | P_{D} | 8.13 (d, J = 9 Hz; 1H), 8.04 (dd, J = 3, |
| | | | | | | 8 Hz; 2H), 7.98-7.94 (m; 4H), 7.89 (s; |
| | | | | | | 2H), 7.87 (d, J = 8 Hz; 1H), 7.69 (d, J = |
| | | | | | | 7 Hz; 1H), 5.73 (m; 1H), 5.43 (m; |
| | | | | | | 1H), 4.61 (bs; 1H), 3.95-3.80 (m; |
| | | | | | | 4H), 3.52 (bs; 1H), 3.20 (t; J = 8 Hz; |
| | | | | | | 2H), 2.30 (t, J = 7 Hz; 2H), 2.04 (t; J = |
| | | | | | | 7 Hz; 2H); 1.94 (bs; 2H), 1.55 (bs; 2H). |
| 2 | O—CO | CH₂—CH₂ | 3 | 3 | P_{D} | 8.35/8.23 (AB-system, J = 9 Hz; 2H), |
| | | | | | | 8.28 (dd, J = 4, 8 Hz; 2H), 8.23 (d, J = |
| | | | | | | 8 Hz; 1H), 8.1 5/8.13 (AB-system, J = |
| | | | | | | 9 Hz; 2H), 8.07 (t, J = 8 Hz; 1H), 7.96 |
| | | | | | | (d, J = 8 Hz, 1H), 7.96 (d, J = 8 Hz; |
| | | | | | | 1H); 7.76 (bs; 2H), 5.71-5.67 (m; 1H), |
| | | | | | | 5.46 (dd, J = 15, 6 Hz; 1H), 4.18 (m; |
| | | | | | | 1H), 4.00 (t; J = 7 Hz; 2H), 3.61 (dd, |
| | | | | | | J = 12, 5 Hz; 1H), 3.47 (dd, J = 11, 8 |
| | | | | | | Hz; 1H), 3.33 (m; 2H), 3.06 (bs; 1H), 2.32 |
| | | | | | | (t, J = 7 Hz; 2H), 2.03 (m; 2H); 1.81 |
| | | | | | | (m; 2H), 1.62 (m; 4H), 1.45 (m; 2H). |
| 3 | O—CO | CH₂—CH₂ | 3 | 7 | P_{D} | Electrospray MS 529.3 (M + H)⁺ ESI + |
| | | | | | | C₃₄H₄₃NO₄, calc 529.02. |

| | | | | | |
|---|---|---|---|---|---|
| 4 | CH₂—CH₂ | O—CO | m + n = 8 | P_{D} | 9.16 (d, J = 9 Hz;1H), 8.51 (d, J = 9 |
| | | | | | Hz; 1H), 8.15-7.80 (m; 7H), 5.78 (bs; |
| | | | | | 1H), 5.42 (bs; 1H), 4.56 (bs; 1H); 4.41 |
| | | | | | (t, J = 9 Hz; 2H); 3.97-3.60 (m; 2H), |
| | | | | | 3.50 (bs; 3H), 1.96 (bs; 2H), 1.84- |
| | | | | | 1.77 (m; 2H), 1.50-1.20 (m; 14H). |
| 5 | CH₂—CH₂ | NH—CO | m + n = 8 | P_{D} | CDCl₃ + DMSO-d_{6:}: 8.55 (d, J = 9 Hz; |
| | | | | | 1H), 8.28-8.04 (m; 8H), 7.60 (bs; 1H), |
| | | | | | 5.80 (dt, J = 7, 15 Hz; 1H), 5.45 (dd, |
| | | | | | J = 6, 15 Hz; 1H), 4.42 (bs; 1H); 3.83- |
| | | | | | 3.75 (m; 2H), 3.57 (dt, J = 7, 6 Hz; |
| | | | | | 2H), 3.20 (bs; 1H), 2.04 (dt, J = 7, 6 |
| | | | | | Hz; 2H), 1.77-1.70 (m; 2H), 1.52-1.26 (m; 14H). |
| 6 | CH₂—CH₂ | NH—SO₂ | m + n = 8 | P_{D} | (CD₃OD): 8.94 (d, J = 9.4 Hz; 1H), |
| | | | | | 8.56 (d, 8.2 Hz; 1H), 8.60-8.00 (m; |
| | | | | | 7H), 5.72 (dt, J = 6.6, 15.3 Hz; 1H), |
| | | | | | 5.34 (dd, J = 6.8, 15.3 Hz; 1H), 4.18 |
| | | | | | (t; J = 5.6 Hz; 1H), 3.69/3.56 (AB- |
| | | | | | System, J = 4.0, 11.6, 8.3 Hz; 2H), |
| | | | | | 3.08 (m; 1H), 2.72 (t, J = 6.7 Hz; 2H), |
| | | | | | 2.93 (m; 2H), 1.23-1.07 (m; 4H) 1.07- |
| | | | | | 0.55 (m; 12H). |
| 7 | CH₂—CH₂ | CH₂—NH | m + n = 7 | D_{D} | (CD₃OD): 8.46 (d, J = 8.5 Hz; 1H), |
| | | | | | 8.27 (d, J = 8.7 Hz; 1H), 8.11 (d, J = |
| | | | | | 7.4 Hz; 1H), 7.51-7.46 (m; 2H), 7.19 |
| | | | | | (d, J = 7.5 Hz; 1H), 5.75 (dt, J = 6.4 |
| | | | | | Hz, 16.2 Hz; 1H), 4.30 (dd, J = 5.4, |
| | | | | | 6.8 Hz; 1H), 4.19 (t, J = 5.3 Hz; 1H), |
| | | | | | 3.69/3.57 (AB-system, J = 11.6 Hz, |
| | | | | | 4.0, 8.4 Hz; 2H), 3.11 (m; 2H), 2.80 |
| | | | | | (s; 6H), 2.73 (t, J = 6.9 Hz; 2H), 2.00 |
| | | | | | (q, J = 6.9 Hz, 2H), 1.33-1.22 (m; 2H), |
| | | | | | 1.22-0.90 (m; 14H). |

### EXAMPLES 8 to 12

### Compounds of Formula

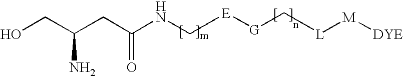

wherein E-G, L-M, m, n and DYE are as defined in TABLE 2:

TABLE 2

| EX | E-G | L-M | m | n | DYE | ¹H-NMR data (d₆-DMSO) |
|---|---|---|---|---|---|---|
| 8 | O—CO | CH₂—CH₂ | 6 | 1 | P_{D} | selected characteristic signals, δ 8.21 |
| | | | | | | (d, J = 9.2 Hz, 1H), 8.10 (d, J = 7 Hz, |
| | | | | | | 1H), 8.05 (d, J = 7 Hz, 1H), 7.77 (d, |
| | | | | | | J = 7.8 Hz, 1H), rest of aromatic signals |
| | | | | | | superposed by acidic protons), 3.98 |
| | | | | | | (t, J = 6.7 Hz, 2H). |
| 9 | CH2—CH₂ | NH—CO | m + n = 8 | | P_{D} | 8.66 (t, J = 5.6 Hz, 1H), 8.45 (d, J = |
| | | | | | | 9.2 Hz, 1H), 8.34 (d, J = 7 Hz, 1H), |
| | | | | | | 8.33 (d, J = 7 Hz, 1H), 8.31 (d, J = 8 |
| | | | | | | Hz, 1H), 8.24 (d, J = 9 Hz, 1H), 8.24 |
| | | | | | | (d, J = 9 Hz, 1H), 8.21 (d, J = 9 Hz, |
| | | | | | | 1H), 8.11 (t, J = 7.6 Hz, 1H), 8.08 (d, |
| | | | | | | J = 8 Hz, 1H), 8.06 (t, J = 5.6 Hz, 1H), |
| | | | | | | 5.22-5.27 (m, 1H), 3.47-3.54 (m, |
| | | | | | | 1H), 3.34-4.43 (m, 4H), 2.97-3.18 |
| | | | | | | (m, 2H), 2.27-2.44 (m, 2H), 1.62 |
| | | | | | | (qui, J = 7.3 Hz, 2H), 1.20-1.44 (m, 14H). |
| 10 | CH₂—CH₂ | NH—SO₂ | m + n = 8 | | P_{D} | 8.98 (d, J = 9.3 Hz, 1H), 8.58 (d, J = |
| | | | | | | 8.2 Hz, 1H), 8.35-8.49 (m, 5H), 8.27 |
| | | | | | | (d, J = 9 Hz, 1H), 8.20 (t, J = 7.7 Hz, |
| | | | | | | 1H), 8.05 (br. s, 2 H), 7.85 (br. s, 3H), |
| | | | | | | 3.47-3.53 (m, 1H), 3.35-3.40 |
| | | | | | | (signals superposed partly by water), |
| | | | | | | 2.92-3.02 (m, 2H), 2.75-2.85 (m, |
| | | | | | | 2H), 2.28-2.56 (signals superposed |
| | | | | | | partly by DMSO), 1.12-1.30 (m, 4H), |
| | | | | | | 0.62-1.05 (m, 12H). |
| 11 | NH—CO | CH₂—CH₂ | 10 | 1 | P_{D} | 8.36 (d, J = 9.3 Hz, 1H), 8.19-8.28 (m, |
| | | | | | | 4H), 8.13 (d, J = 9 Hz, 1H), 8.11 (d, |
| | | | | | | J = 9 Hz, 1H), 8.05 (t, J = 7.6 Hz, 1H), |
| | | | | | | 8.02 (t, J 0 5.6 Hz, 1H), 7.92 (d, J = |
| | | | | | | 7.8 Jz, 1H), 7.79 (t, J = 5.6 Hz, 1H), |
| | | | | | | 5.22 (br. s, 1H), 2.92-3.08 (m, 4H), |
| | | | | | | 2.23-2.40 (m, 2H), 2.20 (t, J = 7.2 Hz, 2H). |

| | | | | | |
|---|---|---|---|---|---|
| 12 | CH₂—CH₂ | CH₂—NH | m + n = 7 | N_{D} | (d6-DMSO): δ 9.52 (br. s, 1H), 8.95 (d, |
| | | | | | J = 8.7 Hz, 1H), 8.12 (t, J = 6 Hz, 1H), |
| | | | | | 7.75 (br. s, 3H), 6.39 (d, J = 8.7 Hz, |
| | | | | | 1H), 3.35-3.65 (signals superposed |
| | | | | | partly by water), 2.94-3.07 (m, 2H), |
| | | | | | 2.30-2.55 (m, 2H), 1.67 (qui, J = 7.2 |
| | | | | | Hz, 2H), 1.20-1.44 (m, 14H). |

### EXAMPLE 13

### Compound of Formula

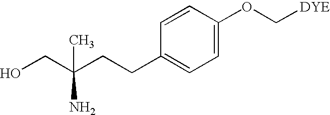

wherein DYE is P_{D}⁻.

¹H-NMR (CDCl₃/d₆-DMSO): selected characteristic signals, δ 8.31 (d, J=9.2 Hz, 1H), 8.05-8.25 (m, 7H), 8.03 (t, J=8.6 Hz, 1H), 7.16 (d, J=8.6 Hz, 2H), 7.02 (d, J=8.6 Hz, 2H), 5.73 (s, 2H), 3.67 (dd, J=12.2+18.1 Hz, 2H), 2.65 (dd, J=6.7+10.6 Hz, 2H), 1.94-2.0 (m, 2H), 1.36 (s, 3H).

### EXAMPLE 14

### (S)-2-Amino-3-[11-(7-nitro-benzo[1,2,5]oxadiazol-4-ylamino)-undecyloxy]-propan-1-ol

### Compound of Formula

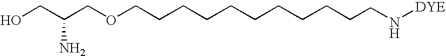

wherein DYE is N_{D}.

A solution of 139 mg of {(S)-1-hydroxymethyl-2-[11-(7-nitro-benzo[1,2,5]oxadiazol-4-ylamino)-undecyloxy]-ethyl}-carbamic acid tert-butyl ester in TFA/H₂O is stirred at RT for 2.5 hours. Solvent is evaporated and the evaporation residue obtained is purified.

(S)-2-Amino-3-[11-(7-nitro-benzo[1,2,5]oxadiazol-4-ylamino)-undecyloxy]-propan-1-ol in the form of a trifluoro acetic acid salt is obtained.

¹H-NMR (DMSO-d₆, 400 MHz) δ: 9.55 (bs; 1H), 8.50 (d; 1H), 7.80 (bs; 3H), 6.40 (d; 1H), 5.20 (bs; 1H), 3.60-3.30 (m; 8H), 3.22 (m; 1H), 1.65 (m; 2H), 1.49 (m; 2H), 1.40-1.15 (m; 14H).

### EXAMPLE C

### Production of STARTING MATERIALS

### C1: {(S)-1-Hydroxymethyl-2-[11-(7-nitro-benzo[1,2,5]oxadiazol-4-ylamino)-undecyloxy]-ethyl}-carbamic acid tert-butyl ester

A solution of 114 mg of [(S)-2-(11-amino-undecyloxy)-1-hydroxymethyl-ethyl]-carbamic acid tert-butyl ester in 6 ml of THF is treated with 76 mg of 7-chloro-4-nitro-benzofurazan and stirred for 16 hours at RT. The reaction mixture obtained is diluted with EtAc and extracted with H₂O, 1N HCl, sat. aq. NaHCO₃ and brine. From the mixture obtained solvent is evaporated, the evaporation residue obtained is purified. {(S)-1-Hydroxymethyl-2-[11-(7-nitro-benzo[1,2,5]oxadiazol-4-ylamino)-undecyloxy]-ethyl}-carbamic acid tert-butyl ester is obtained. Electrospray MS: 564.34 (MNa+).

### C2: [(S)-2-(11-Amino-undecyloxy)-1-hydroxymethyl-ethyl]-carbamic acid tert-butyl ester

Under an atmosphere of argon, a solution of 445 mg of [(S)-2-(11-dibenzylamino-undecyloxy)-1-hydroxymethyl-ethyl]-carbamic acid tert-butyl ester in 18 ml of MeOH is treated with 233 mg of ammonium formiate and 10% Pd on charcoal (57 mg) and stirred at 70° (reflux) for 2 hours. The mixture obtained is cooled to RT, the catalyst is removed by filtration and the filtrate obtained is concentrated. The concentration residue obtained is redissolved in EtAc and extracted with saturated aqueous NaHCO₃ and brine. From the mixture obtained solvent is evaporated and [(S)-2-(11-amino-undecyloxy)-1-hydroxymethyl-ethyl]-carbamic acid tert-butyl ester is obtained. Electrospray MS: 561.3 (MH+).

### C3: [(S)-2-(11-Dibenzylamino-undecyloxy)-1-hydroxymethyl-ethyl]-carbamic acid tert-butyl ester

A solution of 1.42 g of dibenzyl-{11-[(R)-2-(trityl-amino)-3-trityloxy-propoxy]-undecyl}-amine in TFA/H₂O is treated with 1.62 ml of triisopropylsilane and stirred for 16 hours at RT. From the mixture obtained solvent is evaporated and MeOH is added, the precipitate obtained is removed by filtration and the filtration residue obtained is concentrated. The concentration residue obtained is redissolved in diethylether, the mixture obtained is extracted with 1 N NaOH, from the organic phase obtained solvent is evaporated and the evaporation residue obtained is purified. 397 mg of unprotected aminoalcohol are reacted with 289 mg of di-tert. butyldicarbonate in 5 ml of CH₂Cl₂. [(S)-2-(11-Dibenzylamino-undecyloxy)-1-hydroxymethyl-ethyl]-carbamic acid tert-butyl ester is obtained. Electrospray MS: 541.35 (MH+), 563.4 (mNa+).

### C4: Dibenzyl-{11-[(R)-2-(trityl-amino)-3-trityloxy-propoxy]-undecyl}-amine

Under an argon atmosphere a suspension of 1210 mg of KH (20% suspension in paraffin) in 14 ml of toluene is cooled to 4° and treated dropwise with a solution of 1394 mg of (R)-2-(trityl-amino)-3-trityloxy-propanol*. The mixture obtained is stirred for 30 minutes at RT and for 5 minutes at 45° and cooled to 4°. The mixture obtained is treated with a solution of 1354 mg of methanesulfonic acid 11-dibenzylamino-undecyl ester in 6 ml of toluene and the mixture obtained is stirred for 16 hours at 50°. The mixture obtained is cooled to RT and EtAc is added. The organic phase obtained is extracted with aqueous saturated NaHCO₃ and brine, dried and concentrated. Dibenzyl-{11-[(R)-2-(trityl-amino)-3-trityloxy-propoxy]-undecyl}-amine is obtained. Electrospray MS: 925.54 (MH+)

*see e.g. Bartel, M.; Rattay, B.; Nuhn, P. “Synthesis of enantiomerically pure, sn-1 modified sn-2-deoxy-2-amido-glycero-3-phospholipids” Chemistry and Physics of Lipids (2000), 107(1), 121-129.

### C5: Methanesulfonic acid 11-dibenzylamino-undecyl ester

A suspension of 500 mg of 11-amino-undecanol, 738 mg of K₂CO₃ and 698 μl of benzylbromide in 5 ml of DMF is stirred vigorously for 16 hours at RT. From the mixture obtained solvent is evaporated, the evaporation residue obtained is distributed between CH₂Cl₂ and H₂O, the organic layer obtained is dried and concentrated and 11-dibenzylamino-undecanol is obtained. 655 mg of 11-dibenzylamino-undecanol are treated with 252 μl of methansulfonyl chloride in CH₂Cl₂ in the presence of 452 μl of Et₃N at RT. Methanesulfonic acid 11-dibenzylamino-undecyl ester is obtained. Electrospray MS: 446.3 (MH+).

Analogously as described in example 14, but using appropriately protected starting materials compounds of Example 15 to 21 are obtained as set out in the following TABLE 3 and TABLE 4. ¹H-NMR data of the compounds obtained are also indicated in TABLE 3 and TABLE 4.

### EXAMPLE 15 TO 18

Compounds of formula

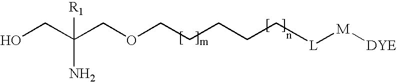

wherein E-G, L-M, m, n and DYE_{EX15} are as defined in TABLE 3:

### R₁ is H for EX 18, R₁ is CH₃ for EX 15 to 17.

TABLE 3

| | | | | ¹H-NMR data |
|---|---|---|---|---|
| EX | L-M | m + n | DYE | (DMSO-d6, 400 MHz) |
| 15 | CH₂—NH | 7 | N_{D} | 9.54 (m; 1H), 8.48 (d; 1H), 7.72 (bs; 3H), 6.40 |
| | | | | (d; 1H), 5.40 (bs; 1H), 3.37 (m; 4H), 1.65/1.48 |
| | | | | (2m; 4H), 1.40-1.20 (m; 14H), 1.10 (s; 3H). |
| 16 | CH₂—NH | 7 | D_{D} | 8.44 (d; 1H), 8.29 (d; 1H), 8.07 (d; 1H), 7.82 (t; |
| | | | | 1H), 7.72 (bs; 3H), 7.57 (m; 2H), 7.22 (d; 1H), |
| | | | | 3.47-3.26 (m; 6H), 2.80 (s; 6H), 2.75 (m; 2H), |
| | | | | 1.48 (m; 2H), 1.30-0.9 (m; 18H), 1.10 (s; 3H). |
| 17 | NH—CO | 8 | P_{D} | (DMSO-d₆, 400 MHz): δ = 8.65 (t; 1H), 8.45 (d; |
| | | | | 1H), 8.33 (m; 3H), 8.23 (m; 3H), 8.10 (m; 2H), |
| | | | | 7.75 (bs; 3H), 5.40 (bs; 1H), 3.36 (m; 6H), 1.62 |
| | | | | (m; 2H), 1.50 (m; 2H), 1.45-1.20 (m; 12H), |
| | | | | 1.11 (s; 3H). |
| 18 | NH—CO | 8 | P_{D} | 8.65 (t; 1H), 8.45 (d; 1H), 8.32 (m; 3H), 8.22 |
| | | | | (m; 3H), 8.11 (m; 2H), 7.75 (bs; 3H), 5.22 (m; |
| | | | | 1H), 3.60-3.15 (m; 9H), 1.68-1.20 (m; 18H). |

### EXAMPLE 19 TO 21

Compounds of formula

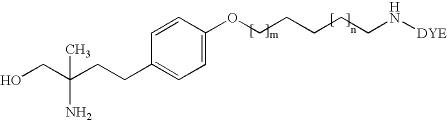

wherein m plus n are as defined in TABLE 4 and DYE is N_{D}.

TABLE 4

| EX | m + n | ¹H-NMR data |
|---|---|---|
| 19 | 5 | (CDCl₃, 400 MHz): δ = 8.48 (d; 1H), 7.90 (bs; 3H), |
| | | 7.06 (m; 3H), 6.77 (d; 2H), 6.15 (d; 1H), 3.90 (t; |
| | | 2H), 3.62 (AB-system; 2H), 3.47 (m; 2H), 2.57 (m; 2H), |
| | | 1.90/1.77 (2m; 4H), 1.55-1.35 (m; 8H), 1.32 (s; 3H). |
| 20 | 8 | (DMSO-d₆, 400 MHz): δ = 9.53 (bs; 1H), 8.48 (d; |
| | | 1H), 7.75 (bs; 3H), 7.05 (d; 2H), 6.81 (d; 2H), |
| | | 6.38 (d; 1H), 5.50 (bs; 1H), 3.86 (t; 2H), 3.43 (m; |
| | | 2H), 2.50 (m; 2H), 1.80-1.60 (m; 6H), 1.42-1.15 (m; |
| | | 14H), 1.18 (s; 3H). |
| 21 | 3 | (CD₃OD, 400 MHz): δ = 8.51 (d; 1H), 7.08 (d; 2H), |
| | | 6.78 (d; 2H), 6.36 (d; 1H), 3.95 (t; 2H), 3.50 (AB- |
| | | system; 2H), 3.55 (m; 2H), 2.62 (m; 2H), 2.00/1.75 |
| | | (m; 6H), 1.55 (m; 4H) 1.34 (s; 3H). |

### EXAMPLE 22

### Phosphoric acid mono-((R)-2-amino-2-methyl-4-{4-[11-(7-nitro-benzo[1,2,5]oxadiazol-4-ylamino)-undecyloxy]-phenyl}-butyl)ester

### Compound of Formula

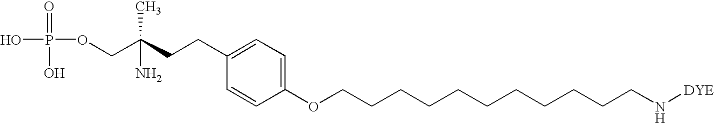

wherein DYE is N_{D}.

A solution of 175 mg of [(R)-1-(di-tert-butoxy-phosphoryloxymethyl)-3-(4-[11-(7-nitro-benzo[1,2,5]oxadiazol-4-ylamino)-undecyloxy]-phenyl)-1-methyl-propyl]-carbamic acid tert-butyl ester in TFA containing 10% H₂O is stirred at RT for 2 hours. From the mixture obtained, solvent is evaporated, to the evaporation residue obtained MeOH is added and the precipitate obtained is collected. Phosphoric acid mono-((R)-2-amino-2-methyl-4-{4-[11-(7-nitro-benzo[1,2,5]oxadiazol-4-ylamino)-undecyloxy]-phenyl}-butyl)ester is obtained.

¹H-NMR (CD₃OD+1 drop DCL, 500 MHz): δ=8.51 (d; 1H), 7.13 (d; 2H), 6.81 (d; 2H), 6.34 (d; 1H), 4.09/4.01 (ABX system, 2H), 3.91 (t; 2H), 3.54 (bs; 2H), 2.63 (m; 2H), 2.04-1.87 (m; 2H), 1.81-1.69 (m; 2H), 1.50-1.29 (m; 14H), 1.41 (s; 3H).

### EXAMPLE D

### Starting Materials

### [(R)-1-Di-tert-butoxy-phosphoryloxymethyl)-3-(4-[1-(7-nitro-benzo[1,2,5]oxadiazol-4-ylamino)-undecyloxy]-phenyl)-1-methyl-propyl]-carbamic acid tert-butyl ester

A solution of 200 mg of [(R)-3-(4-[11-(7-nitro-benzo[1,2,5]oxadiazol-4-ylamino)-undecyloxy]-phenyl)-1-hydroxymethyl-1-methyl-propyl]-carbamic acid tert-butyl ester (see Example 20) and 69 mg of 1H-tetrazole in dry THF are treated with 189 μl of di-tert-butyldiethylphosphoramidite. The mixture obtained is stirred for 105 minutes at RT, 0.33 ml of an aq.30% solution of H₂O₂ in H₂O are added dropwise and stirring is continued for 1 hour. The mixture obtained is distributed between aqueous saturated Na₂S₂O₃ and EtAc. The organic layer obtained is washed with 1 N HCl, saturated NaHCO₃ and brine, dried and concentrated. [(R)-1-(Di-tert-butoxy-phosphoryloxymethyl)-3-(4-[11-(7-nitro-benzo[1,2,5]oxadiazol-4-ylamino)-undecyloxy]-phenyl)-1-methyl-propyl]-carbamic acid tert-butyl ester is obtained. Electrospray MS: 842.49 (MNa+).

### EXAMPLE 23

Phosphoric acid mono-{(R)-2-amino-3-[11-(7-nitro-benzo[1,2,5]oxadiazol-4-ylamino)-undecyloxy]-propyl}ester) is obtained analogously as described in example 22, but using a compound of example 14 as a starting material.

¹H-NMR (CD₃OD+1 drop DCL, 400 MHz): δ=8.57 (d; 1H), 8.35 (bs; 1H), 6.44 (d; 1H), 4.22 (m; 2H), 3.75-3.50 (m; 7H), 1.77 (m; 2H), 1.57 (m; 2H), 1.50-1.23 (m; 14H).

Analogously to a method as described above but using appropriate starting materials, the following compounds of Examples 24 and 25 are obtained;

### EXAMPLE 24

### Compound of Formula

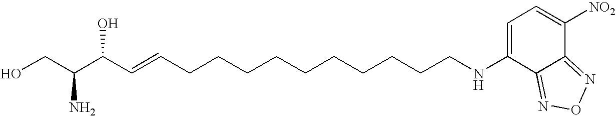

¹H-NMR (CD₃OD, 400 MHz): 8.43 (d, J=9 Hz; 1H), 6.25 (d, J=9 Hz; 1H), 5.74 (dt, J=15, 7 Hz; 1H), 5.36 (dd, J=15, 7 Hz; 1H), 4.18 (dd, J=6, 7 Hz; 1H); 3.69 (dd, 4, 12 Hz; 1H); 3.56 (dd, 8, 12 Hz; 1H); 3.44 (bs; 2H), 3.12-3.03 (m; 1H), 2.00-1.95 (m; 2H), 1.72-1.64 (m; 2H); 1.40-1.25 (m; 14H). Electrospray MS: 436.2 (M+H)+, C21H33N5O5, calcd 435.2).

### EXAMPLE 25

### Compound of Formula

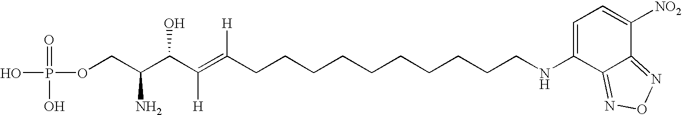

¹H-NMR (CD₃OD, 400 MHz): 8.53 (d, 1H), 6.34 (d, 1H), 5.85 (dt; 1H), 5.46 (dd; 1H), 4.31-4.26 (m; 1H), 4.16-3.98 (m, 2H), 3.57-3.46 (m; 2H), 3.40-3.35 (m; 1H), 2.12-2.05 (m; 2H), 1.81-1.74 (m; 2H); 1.50-1.27 (m; 14H).

### TEST EXAMPLE 1

### Fluorimetric Assay of Sphingosine Kinase Activity

### Assay Principle:

We have determined that sphingosine kinase in the presence of ATP phosphorylates a compound COMPA (compound of Example 23), which is a fluorescent labeled sphingosine, to obtain a corresponding phosphorylated compound COMPA-P (compound of Example 24) according to the Reaction SCHEME 4 as set out below.

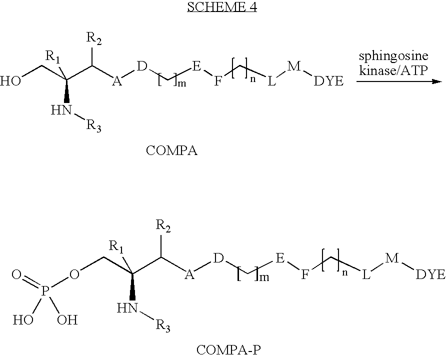

wherein residues are as defined above and R₃ is H. such as phosphorylation of a compound of Example 24 by a sphingosine kinase to obtain a compound of Example 25 in the presence of ATP. COMPA and COMPB are separated by liquid-liquid extraction. Fluorescence as a measure of enzyme activity of a compound of formula COMPA-P in the aqueous phase is quantified. In detail:

Sphingosine kinase is incubated in total volumes of 100 μl with a compound of formula COMPA (20 μM, added from stock solutions in DMSO) and ATP (1 mM) in 50 mM Hepes buffer, pH 7.4, containing 15 mM MgCl₂, 0.005% Triton X-100, 10 mM KCl for 30 minutes at 30° and to the mixture obtained 100 μl 1 M potassium phosphate buffer, pH 8.5, followed by 250 μl CHCl₃/MeOH 2:1 are added. The mixture obtained is briefly mixed and phases are separated by centrifugation (2 min, 15,000×g). An aliquot of the upper aqueous layer (typically 100 μl) is removed and placed into the wells of white 96-well polystyrene microplates (Packard, Meriden, Conn.), followed by addition of an equal volume of DMF.

Fluorescence intensity is measured in a plate reader with excitation at 485 nm and emission at 538 nm. A reaction mixture containing no sphingosine kinase serves as blank. From the fluorescence intensity, concentrations of a compound of formula COMPA-P are calculated using appropriate calibration curves in the same solvent system. These concentrations are also used to calculate reaction rates (phosphate conversion rates) of the enzyme. The assay is suitable to measure sphingosine kinase activity of purified proteins or to determine the activity in lysates of cells or in homogenates of tissues.

### TEST EXAMPLE 2

### Test System

Reaction of Fluorescent Labeled Sphingosines with Sphingosine Kinases

### a) Performance of the Phosphorylation Reaction

The phosphorylation reactions are performed essentially as follows. The cytoplasmic fraction of recombinant HEK-293 cells overexpressing human SPHK-1 or -2 is incubated at 30° in total volumes of 100 μl with SP derivatives (20 μM; added from stock solutions in DMSO), 1 mM of ATP, and 2 μCi [γ-³²P]ATP in 50 mM Hepes buffer (pH 7.4) containing 15 mM MgCl₂, 0.005% Triton X-100, 10 mM KCl , 10 mM NaF and 1.5 mM semicarbazide. Following incubations for different time points up to 2 hours, lipids are extracted and separated by TLC-plates (Merck). TLC is performed on silica plates, using either BuOH/acetic acid/H₂O 3:1:1 or CHCl₃/MeOH/H₂O/NH₄OH (28^{w}/_{w}%) 200:150:29:1 as the mobile phase. In these systems sphingosine and the compound of Example 4 co-migrate. Identity of the metabolites with pyrene-labeled SPP and sphingomyelin (SM) is established by co-migration with commercially available tritium-labeled SPP and SM as standards.

Radiolabeled SPP derivatives are visualized and quantified using a Molecular Dynamics Storm Phosphorlmager (Sunnyvale, Calif.). The rate of phosphorylation is calculated and is reported for the fluorescent labeled sphingosines as value relative to the rate for sphingosine (for which the rate is 41 and 25 nmol/min/mg with SPHK-1 and -2, respectively).

### b) Differences in the Phosphorylation Rate Between Sphingosine Kinase-1 (SPHK-1) and Sphingosine Kinase-2 (SPHK-2)

The fluorescently labeled sphingosines of examples 1-7 are used as substrates for human recombinant SPHK-1 and SPHK-2 and all of them are converted to phosphorylated derivatives as visualized by the incorporation of radiolabeled phosphate upon incubation with [γ-32P]ATP and the kinases. The rates of phosphorylation (phosphate conversion rate) are determined and summarized in Table A relative to the natural substrate sphingosine (=SP), which has the assumed value of 1.

TABLE A

| EX. | SPHK-1 | SPHK-2 |
|---|---|---|
| 1 | 0.048 | 0.14 |
| 2 | 0.078 | 0.83 |
| 3 | 0.024 | 0.065 |
| 4 | 0.83 | 1.25 |
| 5 | 0.28 | 0.59 |
| 6 | 0.051 | 0.83 |
| 7 | 0.063 | 0.026 |

Using SPHK-1, the pyrene-labeled derivative of example 1 with the shortest backbone chain is about 20 times less efficiently converted to the corresponding phosphate than SP. Increasing the chain length by two CH₂-groups (example 2) improves the phosphorylation rate by a factor of 2, whereas further backbone elongation by additional 4 CH₂-groups (example 3) results in inferior substrate turnover by SPHK-1 (about 40-fold less as compared to SP). For a compound of example 4 almost equal phosphorylation by SPHK-1 relative to natural SP is observed. Using SPHK-2, relative phosphorylation rates for compounds of examples 1 to 4 are higher than using SPHK-1, but the ranking of the substrate efficiency is the same for both enzymes.

The ester functionality in a compound of example 4 might be metabolically labile (although no degradation is observed in the short term in vitro phosphorylation experiments) and may, therefore, be replaced by an amide (example 5). This reduces the phosphorylation efficiency only by factor of 2 and 3 for SPHK-1 and -2, respectively. The exchange of the ester to a sulfonamide linker (example 6) is less tolerated by SPHK-1 (phosphate conversion rate about 20 times less than that with a compound of example 4) than by SPHK-2, which phosphorylates a compound of example 6 almost as efficiently as SP. These results indicate pronounced differences in substrate specificity for SPHK-1 versus SPHK-2.

### TEST EXAMPLE 3

### Measurements of Uptake, Subcellular Distribution and Metabolism of Fluorescent Labeled Sphingosines in Human Endothelial Cells

To examine the uptake, subcellular distribution and metabolism (conversion to the phosphate) in human endothelial cells (HUVEC), the pyrene labeled sphingosine of example 4 is used. The fluorescently labeled compound is rapidly incorporated into the cells within 5 minutes after addition, and shows predominantly distribution to the endoplasmic reticulum and to the Golgi apparatus after 15-30 minutes of incubation. Furthermore, the compound is converted intracellularly into 4 products as shown by thin-layer chromatography. The major metabolite co-migrates with sphingosine-1-phosphate (SPP) and a minor product with sphingomyelin (SM) and ceramide (Cer). The pattern of metabolic conversion of the compound in HUVEC is similar to that of [³H]-sphingosine.

### TEST EXAMPLE 4

### Cell Based Assay for the Determination of Sphingosin-1-Phosphate (SPP) and Shingosine-1-Phosphate (SPP) Phosphatase Activities

Human umbilicial vein endothelial cells (HUVEC) are cultured at 37° and 5% CO₂ in EGM™-2 medium (Clonetics; endothelial cell medium). Cells are used for experiments up to passage number 5, seeded in 6-well plates at 80-90% confluency and grown overnight. Cells are incubated with either a labeled compound of formula I wherein X is (HO)₂PO or with a labeled compound of formula I wherein X is H, each at 5 μM for 0.5 hours, 1 hour and 3 hours. The incubation residues obtained after appropriate incubation times are extracted and lipid extraction residues obtained are subjected to TLC in BuOH/AcOH/H₂O as a solvent system. Sphingoid base metabolites as shown in FIG. 1 are detected.

### TEST EXAMPLE 5

### Labeled Sphingosine Conversion in Living Cells in the Presence of a Sphingosine Kinase 1 Inhibitor

HUVEC as described in Test Example 4 are preincubated with a sphingosine kinase 1 inhibitor at 10 μM and 30 μM for 1 hour. A labeled compound of formula I wherein X is H is added and incubation is carried out for 30 minutes. From the incubation residue obtained lipids are extracted and separated by TLC in using BuOH/AcOH/H₂O 3:1:1 as a solvent system. TLC plates obtained are scanned using CCD camera and density of the lipidic products is measured using software AlphaImage 2200.

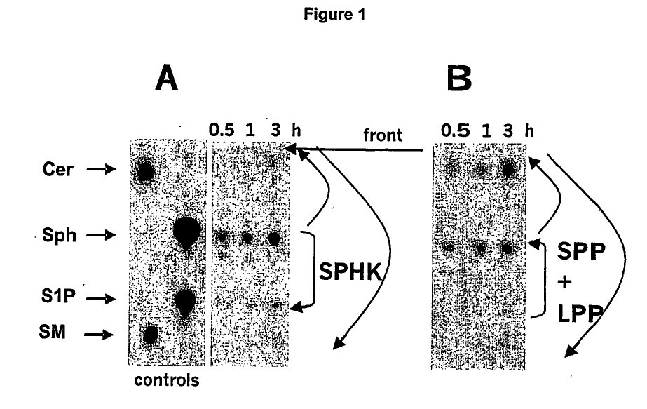

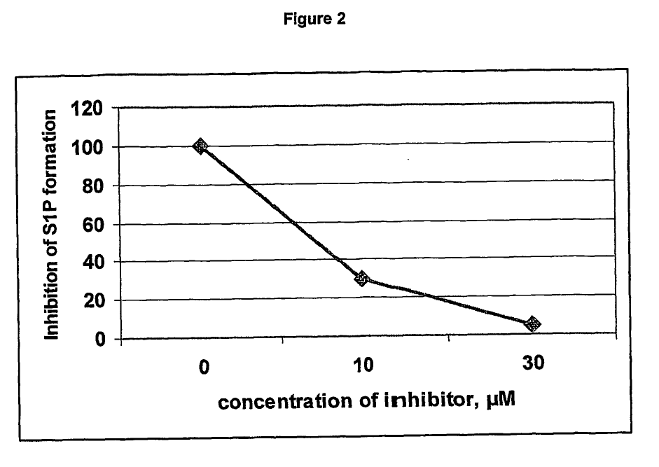

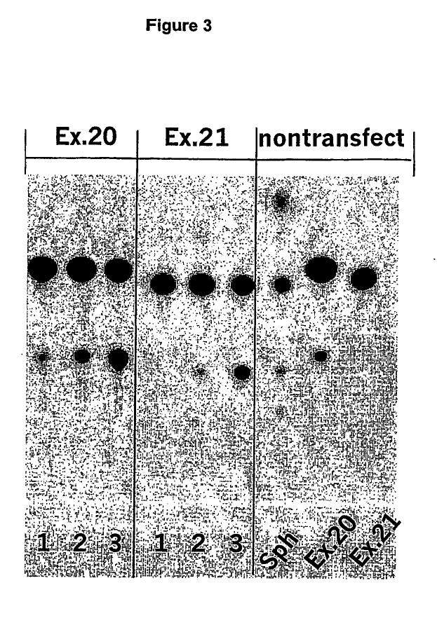

The inhibition curve is shown in FIG. 2.

### TEST EXAMPLE 6

### Sphingolipid Turnover of Labeled Sphingosines in HUVEC Cells which are Transiently Transfected with Sphingosine Kinase Type 1 and 2 (SPHK-1 and SPHK-2)

HUVEC as described in Test Example 4 are seeded in 6-well plates at 50% confluency and grown overnight. Grown cells obtained are transfected using Lipofectamine Plus reagent with an empty vector (GFP-vector from Clontech) alone and GFP-tagged expression constructs coding for SPHK-1 and SPHK-2. Transfection is carried out for 20 hours. To the transfected cells obtained a labeled compound of formula I wherein X is (HO)₂PO is added at 2 μM and incubated for 0.5 hours, 1 hour and 3 hours, respectively. Lipids are extracted and the extraction residue obtained is subjected to TLC using BuOH/AcOH/H₂O 3:1:1 as a solvent system. TLC plates obtained are scanned using CCD camera and density of the lipid products obtained is measured using AlphaImage 2200 (see FIG. 3).

1. A method for determining whether an activity of an enzyme selected from the group consisting of a sphingosine kinase and a phosphatase involved in the sphingolipid pathway is present in a sample or not, or determining the extent of said activity comprising the steps of either
a. contacting living cells comprised in an appropriate culture medium with a labeled sphingosine for a predetermined period of time so that an enzymatic product can be formed,
b. separating the enzymatic product formed in step a., and
c. determining the amount of enzymatic product formed or for determining whether an activity of a sphingosine kinase is present in a sample or not, or determining the extent of said activity comprising the steps of
A. contacting a labeled unphosphorylated sphingosine with
a sample which sample optionally comprises a sphingosine kinase and
a phosphate source,
for a predetermined period of time so that an enzymatic product can be formed,
B. adding to the mixture of step A. an aqueous buffer solution and organic solvent which is able to form two phases in combination with water,
C. separating the phases obtained in step B,
D. determining the amount of enzymatic product in the aqueous phase obtained in step C.

2. A method for identifying an agent that modulates the activity of a sphingosine kinase comprising the steps of
a. contacting a labeled unphsophorylated sphingosine with
a phosphate source, and
a sphingosine kinase
for a predetermined period of time so that an enzymatic product can be formed,
a1. in the absence of a candidate compound, and
a2. in the presence of a candidate compound,
b. adding to the mixture of step a1 and of step a2 an aqueous buffer solution and organic solvent which is able to form two phases in combination with water,
c. separating the unreacted labeled sphingosine from the enzymatic product formed in steps a1. and a2., e.g. according to claim 1, steps b. and c.,
d. detecting the amount of enzymatic product obtained in step a1. and in step a2 and determining whether there is a difference in the amount of enzymatic products formed in step a1. and step a2.,
e. choosing an agent that modulates the activity of a sphingosine kinase as determined in step d.

3. A method for identifying an agent that modulates the activity of a phosphatase involved in the sphingolipid pathway comprising the steps of
A. contacting a labeled phosphorylated sphingosine with living cells comprised in an appropriate medium for a predetermined period of time so that an enzymatic product can be formed,
A1. in the absence of a candidate compound, and
A2. in the presence of a candidate compound,
B. separating the unreacted labeled phosphorylated sphingosine from the enzymatic product formed in steps A1. and A2.,
C. detecting the amount of enzymatic product obtained in step A1. and in step A2 and determining whether there is a difference in the amount of enzymatic products formed in step A1. and step A2.,
D. choosing an agent that modulates the activity of a phosphatase involved in the sphingolipid pathway as determined in step C.

4. A method for determining whether in a sample sphingosine kinase-1-activity or sphingosine kinase-2-activity or both or no sphingosine kinase activity is present comprising the steps of
α. contacting
α1. a labeled unphosphorylated sphingosine with a sample which sample optionally comprises sphingosine kinase-1-activity, or sphingosine kinase-2-activity, or both, or no sphingosine kinase activity, with a phosphate source,
α2. a labeled unphosphorylated sphingosine with a sample comprising a defined amount of sphingosine kinase-1-activity with a phosphate source,
α3. a labeled unphosphorylated sphingosine with a sample comprising a defined amount of sphingosine kinase-2-activity with a phosphate source for a predetermined period of time so that an enzymatic product can be formed,
β. separating the unreacted compound of a labeled sphingosine from the enzymatic product formed in steps α1., α2. and α3., e.g. according to method steps b. and c. as defined in claim 1, and
γ. determining and comparing the phosphate conversion rate in steps α1., α2. and α3.

5. A method for differentiating whether a test compound is capable to mediate the activity of a sphingosine kinase-1 and/or a sphingosine kinase-2 comprising the steps
i. contacting an unphosphorylated compound of formula I with a phosphate source and with
i1. a sphingosine kinase-1,
i2. a sphingosine kinase-2,
in the absence of a test compound, and
in the presence of a test compound
for a predetermined period of time so that an enzymatic product can be formed,
ii. separating the unreacted unphosphorylated compound of formula I from the enzymatic product formed in steps i1. and i2., e.g. according to method steps b. and c. as defined in claim 1, and
iii. determining and comparing the phosphate conversion rate in steps i1. and i2.

6. A kit for kit for determining the activity of an enzyme selected from the group consisting of a sphingosine kinase and a phosphatase involved in the sphingolipid pathway comprising as a main component a labeled sphingosine and instructions for using said kit.

7. A kit of claim 6 for use in the identification of an agent that mediates the activity of an enzyme selected from the group consisting of a sphingosine kinase and a phosphatase involved in the sphingolipid pathway.

8. The method of claim 1, wherein the labeled sphingosine is a compound of formula 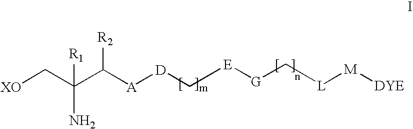
wherein
R₁ is H or (C₁₋₄)alkyl,
R₂ is H, OH or oxo,
X is H or (HO)₂PO,
A-D, E-G and L-M independently of each other is a group CH₂—CH₂, CH═CH, C≡C, CH₂-phenyl, phenyl-CH₂, CH₂—CH₂-phenyl, CH₂—NH, CH₂—N((C₁₋₄)alkyl), NH—CH₂, N((C₁₋₄)alkyl)-CH₂, O—CH₂, CH₂—O, phenyl-O, O-phenyl, CH₂-phenyl-O, O—CO, CO—O, CO—NH, NH—CO, CO—N((C₁₋₄)alkyl), N(C₁₋₄)alkyl)-CO, NH—SO₂, SO₂—NH, N((C₁₋₄)alkyl)-SO₂,
or one group out of A-D, E-G and L-M is absent
m is a number selected from 0 to 12,
n is a number selected from 0 to 12,
and m plus n is a number selected from 0 to 14,
the group DYE is a group selectively detectable in a compound of formula I by physical means, with the proviso that at least one of E-G and L-M is selected from the group consisting of CH₂—NH, CH₂—N((C₁₋₄)alkyl), CH₂—O, phenyl-O, O—CO, CO—O, CO—NH, NH—CO, CO—N((C₁₋₄)alkyl), N(C₁₋₄)alkyl)-CO, NH—SO₂, N((C₁₋₄)alkyl)-SO₂.

9. A compound of formula 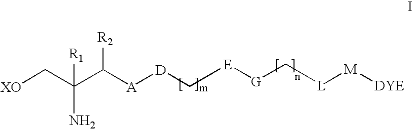
wherein
R₁ is H or (C₁₋₄)alkyl,
R₂ is H, OH or oxo, e.g. H or OH,
X is H or (HO)₂PO,
A-D, E-G and L-M independently of each other is a group CH₂—CH₂, CH═CH, C≡C, CH₂-phenyl, phenyl-CH₂, CH₂—CH₂-phenyl, CH₂—NH, CH₂—N((C₁₋₄)alkyl), NH—CH₂, N((C₁₋₄)alkyl)-CH₂, O—CH₂, CH₂—O, phenyl-O, O-phenyl, CH₂-phenyl-O, O—CO, CO—O, CO—NH, NH—CO, CO—N((C₁₋₄)alkyl), N(C₁₋₄)alkyl)-CO, NH—SO₂, SO₂—NH, N((C₁₋₄)alkyl)-SO₂,
or one group out of A-D, E-G and L-M is absent,
m is a number selected from 0 to 12,
n is a number selected from 0 to 12,
m plus n is a number selected from 0 to 14, and
the group DYE is a group selectively detectable in a compound of formula I by physical means, with the proviso that
at least one of E-G and L-M is selected from the group consisting of CH₂—NH, CH₂—N((C₁₋₄)alkyl), CH₂—O, phenyl-O, O—CO, CO—O, CO—NH, NH—CO, CO—N((C₁₋₄)alkyl), N(C₁₋₄)alkyl)-CO, NH—SO₂, N((C₁₋₄)alkyl)-SO₂,
a compound of formula 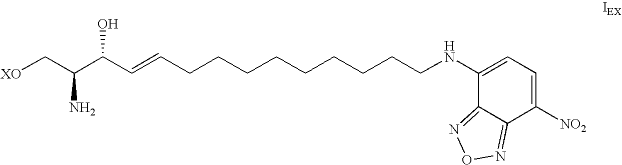
wherein X is as defined above, and
a compound of formula 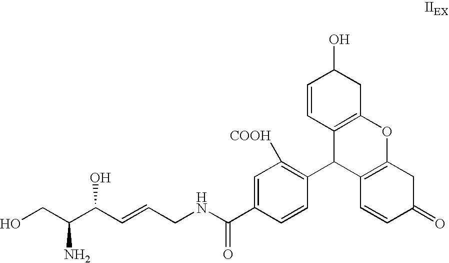
are excluded.

10. The use of a fluorescent labeled sphingosine of formula I as defined in claim 9 in a high-throughput assay, e.g. for the identification of an agent that modulates the activity of an enzyme selected from the group consisting of a sphingosine kinase and a phosphatase involved in the sphingolipid pathway.

11. An agent which is capable to mediate an enzyme selected from the group consisting of a sphingosine kinase and a phosphatase involved in the sphingolipid pathway, which agent is identified by a method of claim 2.
